(19) United States
(12) Patent Application Publication
(10) Pub. No.: US 2014/0322234 A1
(43) Pub. Date: Oct. 30, 2014

(54) Analysis and Targeting of ROR2 in Cancer

(71) Applicant: The Board of Trustees of the Leland Stanford Junior University, Palo Alto, CA (US)

(72) Inventors: Roeland Nusse, Stanford, CA (US); Jan Matthijs Van de Rijn, Stanford, CA (US); Badreddin Edris, Sunnyvale, CA (US)

(21) Appl. No.: 14/364,287

(22) PCT Filed: Jan. 3, 2013
(86) PCT No.: PCT/US2013/020018

§ 371 Date: Jun. 10, 2014

Related U.S. Application Data
(60) Provisional application No. 61/582,713, filed on Jan. 3, 2012.

(51) Int. Cl. G01N 33/574; C12Q 1/68; C07K 16/40

(52) U.S. Cl. CPC G01N 33/57492; C07K 16/40; C12Q 1/6886; G01N 33/57446; G01N 33/57407
USPC 424/158.1; 435/375; 435/7.23; 435/6.11

(57) ABSTRACT

ROR2 is provided as a therapeutic target and prognostic marker for cancers, which include without limitation specific carcinomas and sarcomas. This invention also provides for the use of conjugates comprising an antibody that recognizes and binds ROR2, and a cytotoxic agent. In the cytotoxic conjugates, the cell binding agent has a high affinity for ROR2 and the cytotoxic agent has a high degree of cytotoxicity for cells expressing ROR2, such that the cytotoxic conjugates of the present invention form effective killing agents. In a preferred embodiment, the cell binding agent is an anti-ROR2 antibody or an epitope-binding fragment thereof, more preferably a humanized anti-ROR2 antibody or an epitope-binding fragment thereof, wherein a cytotoxic agent is covalently attached, directly or via a cleavable or non-cleavable linker, to the antibody or epitope-binding fragment thereof.

### GOVERNMENT RIGHTS

This invention was made with Government support under contract CA112270 awarded by the National Institutes of Health. The Government has certain rights in this invention.

### BACKGROUND OF THE INVENTION

A major challenge of cancer treatment is to select specific therapies for distinct tumor types in order to maximize efficacy and minimize toxicity and to provide accurate diagnostic, prognostic, and predictive information.

Sarcomas are a heterogeneous group of over 60 tumour types that originate from mesenchymal cells and that account for approximately 1% of all human malignancies. Most sarcomas demonstrate a propensity for locally aggressive growth and distant haematogenous spread. Leiomyosarcomas (LMS) are malignant tumours of smooth muscle that show a high degree of molecular heterogeneity and are characterized by local recurrence and metastasis; currently, there exist no targeted therapies for LMS. Gastrointestinal stromal tumors (GIST) are thought to arise from the interstitial cells of Cajal in the wall of the gastrointestinal tract and have been shown to respond favourably to treatment with the tyrosine kinase inhibitor imatinib and other small molecule drugs. However, almost all GIST patients eventually develop resistance to treatment, thereby making the exploration of additional therapeutic approaches necessary.

Carcinomas are the most common type of human cancer, arising from cells that have developed the cytological appearance, histological architecture, or molecular characteristics of epithelial cells. Carcinomas are quite heterogeneous entities, reflecting the wide variety, intensity, and potency of various carcinogenic promoters. Subtypes include adenocarcinomas, squamous cell carcinoma, anaplastic carcinoma, large cell and small cell carcinoma, and mixtures thereof. Carcinomas are also classified by the site in which they occur, for example lung cancer, ductal carcinoma of the breast, adenocarcinoma of the prostate, adenocarcinoma or squamous cell carcinoma of the colon and rectum, and the like.

Receptor tyrosine kinases (RTKs) are a family of cell surface receptors that regulate a range of normal cellular processes through ligand-controlled tyrosine kinase activity. Over the past 20 years, deregulation of RTKs has been shown to play critical roles in cancer development and progression. RTKs are now recognized as prognostic molecular biomarkers and as targets of oncology therapeutics.

ROR2 (originally named the “receptor tyrosine kinase-like orphan receptor 2”) is a membrane-bound RTK that is activated by non-canonical Wnt signalling through its association with the Wnt5A glycoprotein during the course of normal bone and cartilage development. ROR2 expression is required to mediate the migration of cells during palate development in mammals and mutations in the ROR2 gene have been shown to cause diseases such as brachydactyly type B and autosomal recessive Robinow syndrome. ROR2 has been reported to have pro-tumorigenic effects in certain cell lines. However, the expression of ROR2, as well as its functional and prognostic significance, has yet to be evaluated in soft-tissue sarcomas and other specific carcinomas. The present invention addresses this issue.

### SUMMARY OF THE INVENTION

ROR2 is provided as a therapeutic and prognostic marker for cancers including, without limitation, carcinomas, e.g. carcinoma of the breast, etc.; and sarcomas, for example leiomyosarcoma (LMS), gastrointestinal stromal tumors (GIST), etc.

For prognostic purposes, particularly in LMS and GIST, tumors that are express ROR2 at a high level relative to a normal control cell are classified as having aggressive tumor growth with a poor patient outcome. The methods of the invention may comprise providing diagnostic, prognostic, or predictive information based on classifying a GIST or LMS as ROR2 positive. For example, this may involve stratifying the tumor (and thus stratifying a subject having the tumor) for a clinical trial. The prognostic methods may further comprise providing an analysis to the patient, and selecting a treatment based on the classifying step.

For therapeutic purposes, antibodies that specifically bind to ROR2 are useful in decreasing or preventing the growth of tumor cells. Such antibodies may act in a variety of modalities, including: blocking the biological activity of ROR2, e.g. preventing ligand binding, altering ROR2 signalling pathways, etc., inducing cell death, e.g. by apoptosis, by antibody-dependent cell-mediated cytotoxicity (ADCC), by complement-dependent cytotoxicity (CDC), etc.; and selective delivery of a toxic conjugate, e.g. chemotherapeutic agent, toxin, radioisotope, etc. Such antibodies are useful in the treatment of cancers including, without limitation, carcinomas, e.g. carcinoma of the breast, etc.; and sarcomas, for example leiomyosarcoma (LMS), gastrointestinal stromal tumors (GIST), etc.

Antibodies useful in the methods of the invention include ROR2-specific monoclonal antibodies, which are optionally chimeric version of murine antibodies, where the constant regions are replaced with human constant region sequences. Also included are resurfaced or humanized versions of such antibodies wherein surface-exposed residues of the variable region frameworks of the antibodies, or their epitope-binding fragments, are replaced in both light and heavy chains to more closely resemble known human antibody surfaces. The humanized antibodies and epitope-binding fragments thereof have a benefit in that they are less immunogenic than murine versions in human subjects to which they are administered. Also encompassed is the use of fragments of anti-ROR2 antibodies that retain the ability to bind ROR2. In another aspect of the invention, the use of functional equivalents of anti-ROR2 antibodies is contemplated.

This invention also provides for the use of conjugates comprising an antibody that recognizes and binds ROR2, and a cytotoxic agent. In the cytotoxic conjugates, the cell binding agent has a high affinity for ROR2 and the cytotoxic agent has a high degree of cytotoxicity for cells expressing ROR2, such that the cytotoxic conjugates of the present invention form effective killing agents. In a preferred embodiment, the cell binding agent is an anti-ROR2 antibody or an epitope-binding fragment thereof, more preferably a humanized anti-ROR2 antibody or an epitope-binding fragment thereof, wherein a cytotoxic agent is covalently attached, directly or via a cleavable or non-cleavable linker, to the antibody or epitope-binding fragment thereof. In some embodiments, the cytotoxic agent is a chemotherapeutic drug.

The present invention also provides a method for inhibiting the growth of a cell expressing ROR2, e.g. a cancer cell, by contacting the cell with an anti-ROR2 antibody or conjugate thereof under conditions permissive for growth inhibition, e.g. in a dose and for a time sufficient to induce cell death, in the presence of complement, etc. In some embodiments, the method for inhibiting the growth of the cell expressing ROR2 takes place in vivo and results in the death of the cell, although in vitro and ex vivo applications are also included. Also included is a method of treating a subject having a cancer using the therapeutic antibody composition.

The present invention also provides a therapeutic composition comprising an anti-ROR2 antibody or an anti-ROR2 antibody-cytotoxic agent conjugate, and a pharmaceutically acceptable carrier or excipients. In some embodiments, the therapeutic composition comprises a second therapeutic agent. The antibody or antibody conjugate or formulation thereof may be provided in a kit with instructions for use. The kit may also include components necessary for the preparation of a pharmaceutically acceptable formulation, such as a diluent if the conjugate is in a lyophilized state or concentrated form, and for the administration of the formulation.

### BRIEF DESCRIPTION OF THE DRAWING

FIG. 1. ROR2 mRNA expression in soft-tissue sarcomas. Expression of ROR2 mRNA in 148 soft-tissue sarcoma cases was evaluated using gene microarrays.

FIG. 2. Representative immunohistochemical stains for ROR2 in LMS and GIST. Samples were scored as follows: 2: strong staining whether diffusely or focally present in the tumour; 1: weak staining whether diffusely or focally present in the tumour; 0: absence of any staining (scale bar, 0.2 mm). Examples of each score are shown for LMS and GIST.

FIG. 3. Effects of in vitro ROR2 downregulation and activation on invasive LMS and GIST cell lines. ROR2 protein expression was analysed by IHC on paraffin-embedded pellets of cell lines (scale bar, 35 μm) in LMS04, LMS05, and GIST48 (A). siROR2 treatment downregulated ROR2 transcript levels and inhibited the invasion of ROR2-positive LMS05 and GIST48 cells through matrigel chambers, whereas no effect is seen in ROR2-negative LMS04 (B, C). GIST48 cells were treated with ROR2-ligand Wnt5A and cell lysates were precipitated with anti-ROR2 antibody and subjected to immunoblotting with anti-phospho-Tyrosine (top) or anti-ROR2 (bottom) antibodies (D). Wnt5A-treated GIST48 whole-cell lysates were subjected to immunoblotting with anti-phospho-Tyrosine (top), anti-ROR2 (middle), or anti-Actin (bottom) antibodies (E). Treatment of ROR2-positive LMS05 and GIST48 with Wnt5A increased cell invasion, an effect that was diminished by siROR2 treatment; ROR2-negative LMS04 showed no response to treatment with Wnt5A (F). All experiments were performed in triplicate; error bars are ±one standard deviation. ** denotes statistical significance at P<0.01 and * denotes statistical significance at P<0.05 as determined by Student's t-test.

FIG. 4. Effects of in vitro ROR2 upregulation on minimally invasive GIST cell line. ROR2 protein expression was analysed by IHC on a paraffin-embedded pellet of the GIST882 cell line (scale bar, 35 μm) (A). Transfection of ROR2 into GIST882 cells resulted in a strong upregulation of ROR2 mRNA and protein (B, C). This resulted in a greater than two-fold increase in the invasive capacity of these cells (D). All experiments were performed in triplicate; error bars are ±one standard deviation. ** denotes statistical significance at P<0.01 as determined by Student's t-test.

FIG. 5. ROR2 downregulation decreases in vivo tumour mass. ROR2 protein expression was analysed by IHC in LMS05 cells that stably expressed a control shRNA or one of two independent ROR2-specific shRNA constructs (A). Cell lines with ROR2-specific shRNAs resulted in significantly reduced tumour masses when grown as subcutaneous xenografts in NSG mice (B). Tumour presence was confirmed by H&E staining (left panel) and ROR2 protein expression was measured by IHC (right panel) in the xenografts (C).

FIG. 6. ROR2 expression predicts poor clinical outcome in patients with GIST and LMS. Kaplan-Meier survival curves for GIST and LMS cases stratified by ROR2 protein expression. High ROR2 expression predicted poor overall survival in patients with GIST (A) and poor disease-specific survival in patients with both gynaecological LMS (B) and non-gynaecological LMS (C).

FIG. 7. ROR2 expression is maintained between primary and metastasis tumours. ROR2 expression was analysed by IHC in a series of primary gynaecological and non-gynaecological LMS, as well as their associated metastases; numbers along the left represent Case IDs (A). Examples of primary-metastasis pairs showing consistently high or low ROR2 expression are shown for gynaecological (top two panels) and non-gynaecological (bottom two panels) LMS cases (B).

FIG. 8. Immunohistochemistry of normal tissue and breast cancer samples.

FIG. 9. Flow cytometry staining with ROR2 of live sarcoma cell lines.

FIG. 10. Treatment with anti-ROR2 mAb resulted in a 40% decrease in tumor mass.

FIGS. 11A-11B. Treatment with ROR2-ligand Wnt5A resulted in an increase in endogenous ROR2 receptor activation as measured by ROR2 phosphorylation, and this activation was markedly diminished in the presence of the anti-ROR2 mAb, as determined by Western blot and quantified by densitometry using the ImageJ software.

### DETAILED DESCRIPTION OF THE EMBODIMENTS

The present application refers to various patents, publications, books, articles, and other references. The contents of all of these items are hereby incorporated by reference in their entirety. In particular, a numbered list of references appears following the Examples, all of which are incorporated herein by reference.

### I. DEFINITIONS

To facilitate understanding of the invention, the following definitions are provided. It is to be understood that, in general, terms not otherwise defined are to be given their meaning or meanings as generally accepted in the art.

Agonist: As used herein, the term “agonist” refers to an agent that increases or prolongs the duration of the effect of a polypeptide or a nucleic acid. Agonists may include proteins, nucleic acids, carbohydrates, lipids, small molecules, ions, or any other molecules that modulate the effect of the polypeptide or nucleic acid. An agonist may be a direct agonist, in which case it is a molecule that exerts its effect by binding to the polypeptide or nucleic acid, or an indirect agonist, in which case it exerts its effect via a mechanism other than binding to the polypeptide or nucleic acid (e.g., by altering expression or stability of the polypeptide or nucleic acid, by altering the expression or activity of a target of the polypeptide or nucleic acid, by interacting with an intermediate in a pathway involving the polypeptide or nucleic acid, etc.)

Antagonist: As used herein, the term “antagonist” refers to an agent that decreases or reduces the duration of the effect of a polypeptide or a nucleic acid. Antagonists may include proteins, nucleic acids, carbohydrates, or any other molecules that modulate the effect of the polypeptide or nucleic acid. An antagonist may be a direct antagonist, in which case it is a molecule that exerts its effect by binding to the polypeptide or nucleic acid, or an indirect antagonist, in which case it exerts its effect via a mechanism other than binding to the polypeptide or nucleic acid (e.g., by altering expression or stability of the polypeptide or nucleic acid, by altering the expression or activity of a target of the polypeptide or nucleic acid, by interacting with an intermediate in a pathway involving the polypeptide or nucleic acid, etc.)

Allelic variant: As used herein, an allelic variant of a parent gene is a naturally occurring variant of a gene that differs from the parent gene by one or possibly two or more mutations. Mutations may include, but are not limited to, deletions, additions, substitutions, and amplification of regions of genomic DNA that include all or part of a gene. Generally, allelic variants differ by a single mutation. Under certain circumstances mutations within a gene may be silent. The term is also used herein to refer to a polypeptide that is encoded by an allelic variant of a parent gene.

Diagnostic information: As used herein, diagnostic information or information for use in diagnosis is any information that is useful in determining whether a patient has a disease or condition and/or in classifying the disease or condition into a phenotypic category or any category having significance with regards to the prognosis of or likely response to treatment (either treatment in general or any particular treatment) of the disease or condition. Similarly, diagnosis refers to providing any type of diagnostic information, including, but not limited to, whether a subject is likely to have a condition (such as a tumor), information related to the nature or classification of a tumor, information related to prognosis and/or information useful in selecting an appropriate treatment. Selection of treatment may include the choice of a particular chemotherapeutic agent or other treatment modality such as surgery, radiation, etc., a choice about whether to withhold or deliver therapy, etc.

Fragment: For the purposes of the present invention, a fragment of a parent polypeptide is a naturally occurring fragment (e.g., a fragment that is produced by digestion with a digestive protease) or a fragment that is characteristic of the polypeptide (e.g., a peptide that is unique to that polypeptide). Generally, a fragment of the present invention will be missing one or more amino acids from the N- and/or C-terminus of the parent polypeptide. A fragment will typically include 20 or more amino acids, preferably 40 or more amino acids.

Gene: For the purposes of the present invention, the term “gene” has its meaning as understood in the art. For the purpose of clarity we note that, as used herein, the term “gene” generally refers to a portion of a nucleic acid that encodes a protein; the term may optionally encompass regulatory sequences. This definition is not intended to exclude application of the term “gene” to non-protein coding expression units but rather to clarify that, in most cases, the term as used in this document refers to a protein coding nucleic acid. It will be appreciated that in the context of the present invention a “gene” as defined herein encompasses any nucleotide molecule that encodes a particular polypeptide (i.e., taking into account possible degeneracies in the genetic code).

Gene product or expression product: A gene product or expression product is, in general, an RNA transcribed from the gene or a polypeptide encoded by an RNA transcribed from the gene.

Marker: A marker, as used herein, refers to a gene whose expression is characteristic of a particular tumor subclass. The term may also refer to a product of gene expression, e.g., an RNA transcribed from the gene or a translation product of such an RNA, the production of which is characteristic of a particular tumor subclass. In some cases expression or levels of a marker may be the sole criterion used to define the tumor subclass. In other cases expression or levels of a marker may be combined with other criteria to define the tumor subclass. The statistical significance of the presence or absence of a marker may vary depending upon the particular marker. In some cases the detection of a marker is highly specific in that it reflects a high probability that the tumor is of a particular subclass. This specificity may come at the cost of sensitivity, i.e., a negative result may occur even if the tumor is a tumor that would be expected to express the marker. Conversely, markers with a high degree of sensitivity may be less specific than those with lower sensitivity. Thus it will be appreciated that a useful marker need not distinguish tumors of a particular subclass with 100% accuracy. Furthermore, it will be appreciated that the use of multiple markers may improve the specificity and/or sensitivity with which a tumor can be identified as being of a particular tumor subclass. It is to be understood that a marker for a particular tumor subclass is a gene (or gene product) whose expression is characteristic of a particular tumor subclass, i.e., a gene (or gene product) whose expression is characteristic of some or all of the cells in the tumor.

Positive or negative subclass: As used herein, a tumor belonging to a positive subclass includes cells with a mutated or upregulated version of a particular marker gene. A tumor belonging to a negative subclass includes cells with a wild-type version or level of expression of a particular marker gene. Mutations may result in overexpression or inappropriate expression of the marker gene. Additionally or alternatively mutations may result in an overly activated gene product (e.g., polypeptide).

Prognostic and predictive information: As used herein the terms prognostic and predictive information are used interchangeably to refer to any information that may be used to foretell any aspect of the course of a disease or condition either in the absence or presence of treatment. Such information may include, but is not limited to, the average life expectancy of a patient, the likelihood that a patient will survive for a given amount of time (e.g., 6 months, 1 year, 5 years, etc.), the likelihood that a patient will be cured of a disease, the likelihood that a patient's disease will respond to a particular therapy (wherein response may be defined in any of a variety of ways). Prognostic and predictive information are included within the broad category of diagnostic information.

Response: As used herein a response to treatment may refer to any beneficial alteration in a subject's condition that occurs as a result of treatment. Such alteration may include stabilization of the condition (e.g., prevention of deterioration that would have taken place in the absence of the treatment), amelioration of symptoms of the condition, improvement in the prospects for cure of the condition, etc. One may refer to a subject's response or to a tumor's response. In general these concepts are used interchangeably herein.

Tumor or subject response may be measured according to a wide variety of criteria, including clinical criteria and objective criteria. Techniques for assessing response include, but are not limited to, clinical examination, chest X-ray, CT scan, MRI, ultrasound, endoscopy, laparoscopy, presence or level of tumor markers in a sample obtained from a subject, cytology, histology. Many of these techniques attempt to determine the size of a tumor or otherwise determine the total tumor burden. The exact response criteria can be selected in any appropriate manner, provided that when comparing groups of tumors and/or patients, the groups to be compared are assessed based on the same or comparable criteria for determining response rate. One of ordinary skill in the art will be able to select appropriate criteria.

Sample: As used herein, a sample obtained from a subject may include, but is not limited to, any or all of the following: a cell or cells, a portion of tissue, blood, serum, ascites, urine, saliva, and other body fluids, secretions, or excretions. The term “sample” also includes any material derived by processing such a sample. Derived samples may include nucleotide molecules or polypeptides extracted from the sample or obtained by subjecting the sample to techniques such as amplification or reverse transcription of mRNA, etc.

Specific binding: As used herein, the term refers to an interaction between a target polypeptide (or, more generally, a target molecule) and a binding agent such as an antibody. The interaction is typically dependent upon the presence of a particular structural feature of the target molecule such as an antigenic determinant or epitope recognized by the binding molecule. For example, if an antibody is specific for epitope A, the presence of a polypeptide containing epitope A or the presence of free unlabeled A in a reaction containing both free labeled A and the antibody thereto, will reduce the amount of labeled A that binds to the antibody. It is to be understood that specificity need not be absolute. For example, it is well known in the art that numerous antibodies cross-react with other epitopes in addition to those present in the target molecule. Such cross-reactivity may be acceptable depending upon the application for which the antibody is to be used. One of ordinary skill in the art will be able to select antibodies having a sufficient degree of specificity to perform appropriately in any given application, e.g. for detection of a target molecule, for therapeutic purposes, etc. It is also to be understood that specificity may be evaluated in the context of additional factors such as the affinity of the binding molecule for the target molecule versus the affinity of the binding molecule for other targets, e.g., competitors. If a binding molecule exhibits a high affinity for a target molecule that it is desired to detect and low affinity for non-target molecules, the antibody will likely be an acceptable reagent for immunodiagnostic purposes. Once the specificity of a binding molecule is established in one or more contexts, it may be employed in other, preferably similar, contexts without necessarily re-evaluating its specificity.

Treating a tumor: As used herein, treating a tumor is taken to mean treating a subject who has the tumor.

Tumor subclass: A tumor subclass is the group of tumors that display one or more phenotypic or genotypic characteristics that distinguish members of the group from other tumors.

Tumor sample: The term “tumor sample” as used herein is taken broadly to include cell or tissue samples removed from a tumor, cells (or their progeny) derived from a tumor that may be located elsewhere in the body (e.g., cells in the bloodstream or at a site of metastasis), or any material derived by processing such a sample. Derived tumor samples may include nucleic acids or proteins extracted from the sample or obtained by subjecting the sample to techniques such as amplification or reverse transcription of mRNA, etc.

### Gastrointestinal Stromal Tumors:

GISTs occur in the wall of the bowel and have been proposed to arise from the interstitial cells of Cajal. The differential diagnosis of these tumors includes desmoid fibromatosis, Schwannoma, leiomyosarcoma, and, in some cases, high grade sarcomas. Accurate diagnosis of GISTs is important, because imatinib mesylate has been shown to significantly inhibit these tumors. In some embodiments, an individual suspected of having a GIST is assessed by the methods of the invention for increased expression of ROR2.

Currently, the diagnosis of GISTs relies heavily on expression of the KIT marker. Recommendations in the literature emphasize a diffuse, strong KIT immunoreactivity for the diagnosis of GIST, CD34 immunostaining can also aid in the diagnosis, but a subset of cases is immunonegative while many other types of sarcomas are immunoreactive for this marker. In the vast majority of GISTs, high levels of KIT expression are accompanied by a KIT mutation in exon 9, 11, 13 or 17. Recently, a subset of GISTs have been found to have PDGFRA mutations rather than KIT mutations. Patients with GISTs containing mutations in PDGFRA belong to the PDGFRA positive subclass and may still benefit from imatinib therapy. However, this subclass of tumors often fail to react with antibodies against KIT and hence may remain undiagnosed as GISTs. Furthermore, identification of PDGFRA positive mutant GISTs currently requires molecular analysis, a laborious process that is not ideal for application in a routine clinical setting. In addition, some GISTs with KIT mutations may have low KIT expression by immunohistochemistry yet will still respond to imatinib therapy. There is therefore an urgent need for methods of identifying and classifying these hard to detect subclasses of GISTs.

Carcinoma of the breast. Breast cancer most often involves glandular breast cells in the ducts or lobules. Most patients present with an asymptomatic lump discovered during examination or screening mammography, and diagnosis is confirmed by biopsy. Treatment usually includes surgical excision, often with radiation therapy, with or without adjuvant chemotherapy, hormonal therapy, or both. About 5% of women with breast cancer carry a mutation in one of the 2 known breast cancer genes, BRCA1 or BRCA2. If relatives of such a woman also carry the gene, they have a 50 to 85% lifetime risk of developing breast cancer. Women with BRCA1 mutations also have a 20 to 40% lifetime risk of developing ovarian cancer; risk among women with BRCA2 mutations is increased less. An individual with breast cancer suspected of having a ROR2 posivie breast cancer may be assessed for the ROR2 phenotype of the tumor, and treated with a ROR2 antibody if appropriate, i.e. the carcinoma overexpresses ROR2.

Most breast cancers are epithelial tumors that develop from cells lining ducts or lobules; less common are nonepithelial cancers of the supporting stroma (e.g. angiosarcoma, primary stromal sarcomas, phyllodes tumor). Cancers are divided into carcinoma in situ and invasive cancer. Carcinoma in situ is proliferation of cancer cells within ducts or lobules and without invasion of stromal tissue. Usually, ductal carcinoma in situ (DCIS) is detected only by mammography and is localized to one area; it may become invasive. Lobular carcinoma in situ (LCIS) is a nonpalpable lesion usually discovered via biopsy; it is rarely visualized with mammography. LCIS is often multifocal and bilateral. It is not malignant, but its presence indicates increased risk of subsequent invasive carcinoma in either breast. Invasive carcinoma is primarily adenocarcinoma. About 80% is the infiltrating ductal type; most of the remaining cases are infiltrating lobular. Rare types include medullary, mucinous, and tubular carcinomas.

Breast cancer invades locally and spreads initially through the regional lymph nodes, bloodstream, or both. Metastatic breast cancer may affect almost any organ in the body—most commonly, lungs, liver, bone, brain, and skin.

Estrogen and progesterone receptors are present in some breast cancers. About two thirds of postmenopausal patients have an estrogen-receptor positive (ER+) tumor. Another cellular receptor is human epidermal growth factor receptor 2 (HER2; also, HER2/neu or ErbB2); its presence correlates with a poorer prognosis at any given stage of cancer.

Antibody or “antibody moiety” is intended to include any polypeptide chain-containing molecular structure that has a specific shape which fits to and recognizes an epitope, where one or more non-covalent binding interactions stabilize the complex between the molecular structure and the epitope. The archetypal antibody molecule is the immunoglobulin, and all types of immunoglobulins (IgG, IgM, IgA, IgE, IgD, etc.), from all sources (e.g., human, rodent, rabbit, cow, sheep, pig, dog, other mammal, chicken, turkey, emu, other avians, etc.) are considered to be “antibodies.” Antibodies utilized in the present invention may be polyclonal antibodies, although monoclonal antibodies are preferred because they may be reproduced by cell culture or recombinantly, and may be modified to reduce their antigenicity. Methods of raising antibodies and generating monoclonal antibodies are known to those of skill in the art. Antibodies or antigen binding fragments may also be produced by genetic engineering.

Antibodies that have a reduced propensity to induce a violent or detrimental immune response in humans (such as anaphylactic shock), and which also exhibit a reduced propensity for priming an immune response which would prevent repeated dosage with the antibody therapeutic or imaging agent (e.g., the human-anti-murine-antibody “HAMA” response), are preferred for therapeutic use. Thus, humanized, chimeric, or xenogenic human antibodies, which produce less of an immune response when administered to humans, are preferred for use in the present invention. Alternatively, single chain antibodies (Fv, as described below) can be produced from phage libraries containing human variable regions.

In addition to entire immunoglobulins (or their recombinant counterparts), immunoglobulin fragments comprising the epitope binding site (e.g., Fab′, F(ab′)₂, or other fragments) are useful as antibody moieties in the present invention. Such antibody fragments may be generated from whole immunoglobulins by ficin, pepsin, papain, or other protease cleavage. “Fragment,” or minimal immunoglobulins may be designed utilizing recombinant immunoglobulin techniques. For instance “Fv” immunoglobulins for use in the present invention may be produced by linking a variable light chain region to a variable heavy chain region via a peptide linker (e.g., poly-glycine or another sequence which does not form an alpha helix or beta sheet motif.

Also included within the scope of the invention are functional equivalents of the anti-ROR2 antibody and the humanized anti-ROR2 receptor antibody. The term “functional equivalents” includes antibodies with homologous sequences, chimeric antibodies, artificial antibodies and modified antibodies, for example, wherein each functional equivalent is defined by its ability to bind to the ROR2 protein. The skilled artisan will understand that there is an overlap in the group of molecules termed “antibody fragments” and the group termed “functional equivalents.” Methods of producing functional equivalents are known to the person skilled in the art. Artificial antibodies include scFv fragments, diabodies, triabodies, tetrabodies and mru, each of which has antigen-binding ability. In the single chain Fv fragment (scFv), the V_{H} and VL domains of an antibody are linked by a flexible peptide. Typically, this linker peptide is about 15 amino acid residues long. If the linker is much smaller, for example 5 amino acids, diabodies are formed, which are bivalent scFv dimers. If the linker is reduced to less than three amino acid residues, trimeric and tetrameric structures are formed that are called triabodies and tetrabodies. The smallest binding unit of an antibody is a CDR, typically the CDR2 of the heavy chain which has sufficient specific recognition and binding that it can be used separately. Such a fragment is called a molecular recognition unit or mru. Several such mrus can be linked together with short linker peptides, therefore forming an artificial binding protein with higher avidity than a single mru.

The functional equivalents of the present application also include modified antibodies, e.g., antibodies modified by the covalent attachment of any type of molecule to the antibody. For example, modified antibodies include antibodies that have been modified, e.g., by glycosylation, acetylation, pegylation, phosphorylation, amidation, derivatization by known protecting/blocking groups, proteolytic cleavage, linkage to a cellular ligand or other protein, etc. The covalent attachment does not prevent the antibody from generating an anti-idiotypic response. These modifications may be carried out by known techniques, including, but not limited to, specific chemical cleavage, acetylation, formylation, metabolic synthesis of tunicamycin, etc. Additionally, the modified antibodies may contain one or more non-classical amino acids.

Functional equivalents may be produced by interchanging different CDRs on different chains within different frameworks. Thus, for example, different classes of antibody are possible for a given set of CDRs by substitution of different heavy chains, whereby, for example, IgG1-4, IgM, IgA1-2, IgD, IgE antibody types and isotypes may be produced. Similarly, artificial antibodies within the scope of the invention may be produced by embedding a given set of CDRs within an entirely synthetic framework.

Functional equivalents may be readily produced by mutation, deletion and/or insertion within the variable and/or constant region sequences that flank a particular set of CDRs, using a wide variety of methods known in the art. The antibody fragments and functional equivalents of the present invention encompass those molecules with a detectable degree of binding to ROR2.

The CDRs are of primary importance for epitope recognition and antibody binding. However, changes may be made to the residues that comprise the CDRs without interfering with the ability of the antibody to recognize and bind its cognate epitope. For example, changes that do not affect epitope recognition, yet increase the binding affinity of the antibody for the epitope may be made. Thus, also included in the scope of the present invention are improved versions of both the murine and humanized antibodies, which also specifically recognize and bind ROR2, preferably with increased affinity.

Several studies have surveyed the effects of introducing one or more amino acid changes at various positions in the sequence of an antibody, based on the knowledge of the primary antibody sequence, on its properties such as binding and level of expression (Yang, W. P. et al., 1995, J. Mol. Biol., 254: 392-403; Rader, C. et al., 1998, Proc. Natl. Acad. Sci. USA, 95: 8910-8915; Vaughan, T. J. et al., 1998, Nature Biotechnology, 16: 535-539).

In these studies, equivalents of the primary antibody have been generated by changing the sequences of the heavy and light chain genes in the CDR1, CDR2, CDR3, or framework regions, using methods such as oligonucleotide-mediated site-directed mutagenesis, cassette mutagenesis, error-prone PCR, DNA shuffling, or mutator-strains of E. coli (Vaughan, T. J. et al., 1998, Nature Biotechnology, 16: 535-539; Adey, N. B. et al., 1996, Chapter 16, pp. 277-291, in “Phage Display of Peptides and Proteins”, Eds. Kay, B. K. et al., Academic Press). These methods of changing the sequence of the primary antibody have resulted in improved affinities of the secondary antibodies (Gram, H. et al., 1992, Proc. Natl. Acad. Sci USA, 89: 3576-3580; Boder, E. T. et al., 2000, Proc. Natl. Acad. Sci. USA, 97: 10701-10705; Davies, J. and Riechmann, L., 1996, Immunotechnology, 2: 169-179; Thompson, J. et al., 1996, J. Mol. Biol., 256: 77-88; Short, M. K. et al., 2002, J. Biol. Chem., 277: 16365-16370; Furukawa, K. et al., 2001, J. Biol. Chem., 276: 27622-27628). By a similar directed strategy of changing one or more amino acid residues of the antibody, the antibody sequences described in this invention can be used to develop anti-ROR2 antibodies with improved functions, including improved affinity for ROR2.

Preferred amino acid substitutions are those which: (1) reduce susceptibility to proteolysis, (2) reduce susceptibility to oxidation, (3) alter binding affinity for forming protein complexes, and (4) confer or modify other physico-chemical or functional properties of such analogs. Analogs can include various muteins of a sequence other than the naturally-occurring peptide sequence. For example, single or multiple amino acid substitutions (preferably conservative amino acid substitutions) may be made in the naturally-occurring sequence (preferably in the portion of the polypeptide outside the domain (s) forming intermolecular contacts. A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et al., 1991, Nature, 354: 105, which are each incorporated herein by reference.

Improved antibodies also include those antibodies having improved characteristics that are prepared by the standard techniques of animal immunization, hybridoma formation and selection for antibodies with specific characteristics.

In addition, derivatized immunoglobulins with added chemical linkers, detectable moieties, e.g. fluorescent dyes, enzymes, substrates, chemiluminescent moieties, or specific binding moieties such as streptavidin, avidin, or biotin may be utilized in the methods and compositions of the present invention.

The antibodies can have utility without conjugation, acting to inhibit the growth of tumor cells. However, the cytotoxic effect may be enhanced by conjugation with a cytotoxic moiety; and for imaging purposes it is desirable to conjugate antibodies to an imaging moiety.

As used herein, “cytotoxic moiety” means a moiety which inhibits cell growth or promotes cell death when proximate to or absorbed by the cell. Suitable cytotoxic moieties in this regard include radioactive isotopes (radionuclides), chemotoxic agents such as differentiation inducers and small chemotoxic drugs, toxin proteins such as saporin, and derivatives thereof. As utilized herein, “imaging moiety” means a moiety that can be utilized to increase contrast between a tumor and the surrounding healthy tissue in a visualization technique (e.g., radiography, positron-emission tomography, magnetic resonance imaging, direct or indirect visual inspection). Thus, suitable imaging moieties include radiography moieties (e.g. heavy metals and radiation emitting moieties), positron emitting moieties, magnetic resonance contrast moieties, and optically visible moieties (e.g., fluorescent or visible-spectrum dyes, visible particles, etc.).

Therapeutic or imaging agents may be conjugated to the antibody by any suitable technique, with appropriate consideration of the need for pharmacokinetic stability and reduced overall toxicity to the patient. A therapeutic agent may be coupled to a suitable antibody moiety either directly or indirectly (e.g. via a linker group). For example, a nucleophilic group, such as an amino or sulfhydryl group, may be capable of reacting with a carbonyl-containing group, such as an anhydride or an acid halide, or with an alkyl group containing a good leaving group (e.g., a halide). Alternatively, a suitable chemical linker group may be used. A linker group can function as a spacer to distance an antibody from an agent in order to avoid interference with binding capabilities. A linker group can also serve to increase the chemical reactivity of a substituent on a moiety or an antibody, and thus increase the coupling efficiency. An increase in chemical reactivity may also facilitate the use of moieties, or functional groups on moieties, which otherwise would not be possible.

Suitable linkage chemistries include maleimidyl linkers and alkyl halide linkers (which react with a sulfhydryl on the antibody moiety) and succinimidyl linkers (which react with a primary amine on the antibody moiety). Several primary amine and sulfhydryl groups are present on immunoglobulins, and additional groups may be designed into recombinant immunoglobulin molecules. It will be evident to those skilled in the art that a variety of bifunctional or polyfunctional reagents, both homo- and hetero-functional (such as those described in the catalog of the Pierce Chemical Co., Rockford, Ill.), may be employed as a linker group. Coupling may be effected, for example, through amino groups, carboxyl groups, sulfhydryl groups or oxidized carbohydrate residues. As an alternative coupling method, cytotoxic or imaging moieties may be coupled to the antibody moiety through a an oxidized carbohydrate group at a glycosylation site. Yet another alternative method of coupling the antibody moiety to the cytotoxic or imaging moiety is by the use of a non-covalent binding pair, such as streptavidin/biotin, or avidin/biotin. In these embodiments, one member of the pair is covalently coupled to the antibody moiety and the other member of the binding pair is covalently coupled to the cytotoxic or imaging moiety.

Where a cytotoxic moiety is more potent when free from the antibody portion, it may be desirable to use a linker group that is cleavable during or upon internalization into a cell, or which is gradually cleavable over time in the extracellular environment. A number of different cleavable linker groups have been described. The mechanisms for the intracellular release of a cytotoxic moiety agent from these linker groups include cleavage by reduction of a disulfide bond, by irradiation of a photolabile bond, by hydrolysis of derivatized amino acid side chains, by serum complement-mediated hydrolysis, and acid-catalyzed hydrolysis, etc.

It may be desirable to couple more than one cytotoxic and/or imaging moiety to an antibody. By poly-derivatizing the antibody, several cytotoxic strategies may be simultaneously implemented, an antibody may be made useful as a contrasting agent for several visualization techniques, or a therapeutic antibody may be labeled for tracking by a visualization technique. In one embodiment, multiple molecules of an imaging or cytotoxic moiety are coupled to one antibody molecule. In another embodiment, more than one type of moiety may be coupled to one antibody. Regardless of the particular embodiment, immunoconjugates with more than one moiety may be prepared in a variety of ways. For example, more than one moiety may be coupled directly to an antibody molecule, or linkers that provide multiple sites for attachment (e.g., dendrimers) can be used. Alternatively, a carrier with the capacity to hold more than one cytotoxic or imaging moiety can be used.

Radionuclides for use as cytotoxic moieties are radionuclides which are suitable for pharmacological administration. Such radionuclides include ¹²³I, ¹²⁵I, ¹³¹I, ⁹⁰Y, ²¹¹At, ⁶⁷Cu, ¹⁸⁶Re, ¹⁸⁸Re, ²¹²Pb, and ²¹²Bi. ¹³¹I is particularly preferred, as are other β-radiation emitting nuclides, which have an effective range of several millimeters. ¹²³I, ¹²⁵I, ¹³¹I, or ²¹¹At may be conjugated to antibody moieties for use in the compositions and methods utilizing any of several known conjugation reagents, including Iodogen, N-succinimidyl 3-[²¹¹At]astatobenzoate, N-succinimidyl 3-[¹³¹I]iodobenzoate (SIB), and, N-succinimidyl 5-[¹³¹I]iodob-3-pyridinecarboxylate (SIPC). Any iodine isotope may be utilized in the recited iodo-reagents. Other radionuclides may be conjugated to antibody moieties by suitable chelation agents known to those of skill in the nuclear medicine arts.

Chemotoxic agents include small-molecule drugs such as carboplatin, cisplatin, vincristine, taxanes such as paclitaxel and doceltaxel, hydroxyurea, gemcitabine, vinorelbine, irinotecan, tirapazamine, matrilysin, methotrexate, pyrimidine and purine analogs, and other suitable small toxins known in the art. Chemotoxin differentiation inducers include phorbol esters and butyric acid. Chemotoxic moieties may be directly conjugated to the antibody via a chemical linker, or may encapsulated in a carrier, which is in turn coupled to the antibody.

Toxin proteins for use as cytotoxic moieties include ricins A and B, abrin, diphtheria toxin, bryodin 1 and 2, momordin, trichokirin, cholera toxin, gelonin, Pseudomonas exotoxin, Shigella toxin, pokeweed antiviral protein, saporin, and other toxin proteins known in the medicinal biochemistry arts. As these toxin agents may elicit undesirable immune responses in the patient, especially if injected intravascularly, they may be encapsulated in a carrier for coupling to the antibody.

Radiographic moieties for use as imaging moieties include compounds and chelates with relatively large atoms, such as gold, iridium, technetium, barium, thallium, iodine, and their isotopes. It is preferred that less toxic radiographic imaging moieties, such as iodine or iodine isotopes, be utilized in the compositions and methods of the invention. Such moieties may be conjugated to the antibody through an acceptable chemical linker or chelation carrier. Suitable radionuclides for conjugation include ⁹⁹Tc, ¹¹¹In, and ⁶⁷Ga. Positron emitting moieties for use in the present invention include ¹⁸F, which can be easily conjugated by a fluorination reaction with the antibody.

### Prognostic Methods

According to one aspect, the invention provides a method comprising providing a tumor sample; detecting expression or activity of a gene encoding a ROR2 polypeptide in the sample; and classifying the tumor as a gastrointestinal stromal tumor or leiomyosarcoma belonging to a ROR2 positive subclass based on the results of the detecting step, where the subclass is indicative of a prognosis for poor patient outcome. In certain embodiments, the method further comprises detecting presence of c-Kit polypeptide, or expression or activity of a gene encoding c-Kit polypeptide in the sample and/or detecting presence of PDGFRA polypeptide, or expression or activity of a gene encoding a PDGFRA polypeptide in the sample. According to such embodiments, the classifying step is based on the results of the combined detecting steps. The methods of the invention may further comprise providing diagnostic, prognostic, or predictive information based on classifying a GIST or LMS as ROR2 positive. For example, this may involve stratifying the tumor (and thus stratifying a subject having the tumor) for a clinical trial. The methods may further comprise selecting a treatment based on the classifying step.

In another aspect, the invention provides a method comprising providing a tumor sample; detecting expression or activity of a ROR2 polypeptide in the sample; and classifying the tumor as a gastrointestinal stromal tumor or leiomyosarcoma belonging to a ROR2 positive subclass based on the results of the detecting step, where the subclass is indicative of a prognosis for poor patient outcome. In certain embodiments, the method further comprises detecting expression or activity of a gene encoding a KIT polypeptide in the sample and/or detecting expression or activity of a gene encoding a PDGFRA polypeptide in the sample. According to such embodiments, the classifying step is based on the results of the combined detecting steps. The methods of the invention may comprise providing diagnostic, prognostic, or predictive information based on classifying a GIST or LMS as ROR2 positive. For example, this may involve stratifying the tumor (and thus stratifying a subject having the tumor) for a clinical trial. The methods may further comprise selecting a treatment based on the classifying step.

In any of the above methods, the tumor sample may be a blood sample, a urine sample, a serum sample, an ascites sample, a saliva sample, a cell, or a portion of tissue. As described in greater detail below, the methods may further comprise providing diagnostic, prognostic, or predictive information based on the classifying step. For example a report may be provided assessing the patient risk based on the ROR2 expression profiling. Classifying may include stratifying the tumor (and thus stratifying a subject having the tumor), e.g., for a clinical trial. In certain embodiments, the methods may further comprise selecting a treatment based on the classifying step.

As is well known in the art, a polypeptide may be detected using any of a variety of techniques and binding agents. Any such technique and agent may be used according to the present invention. In certain preferred embodiments, the binding agent is an antibody that binds specifically to the polypeptide. The invention also encompasses the use of protein arrays, including antibody arrays, for detection of a polypeptide, or tissue microarrays. Other types of protein arrays are known in the art. In general, antibodies that bind specifically to an inventive polypeptide may be generated by methods well known in the art and described, for example, in Harlow, E, Lane, E, and Harlow, E, (eds.) Using Antibodies: A Laboratory Manual, Cold Spring Harbor Laboratory Press, Cold Spring Harbor, 1998. Antibodies include, but are not limited to, polyclonal, monoclonal, chimeric (e.g., “humanized”), single chain antibodies, Fab fragments, antibodies generated using phage display technology, etc.

In addition, in certain embodiments of the invention the polypeptides are detected using other specific binding agents known in the art for the detection of polypeptides, such as aptamers (Aptamers, Molecular Diagnosis, Vol. 4, No. 4, 1999), reagents derived from combinatorial libraries for specific detection of proteins in complex mixtures, random peptide affinity reagents, etc. In general, any appropriate binding agent for detecting a polypeptide may be used in conjunction with the present invention, although antibodies may represent a particularly appropriate modality.

In certain embodiments of the inventive methods a single binding agent (e.g., antibody) is used whereas in other embodiments of the invention multiple binding agents, directed either against the same or against different polypeptides can be used to increase the sensitivity or specificity of the detection technique or to provide more detailed information than that provided by a single binding agent. Thus the invention encompasses the use of a battery of binding agents that bind to polypeptides encoded by the marker genes identified herein. These agents can also be used in conjunction with binding agents against polypeptides encoded by other useful marker genes (e.g., CD34, KIT, PDGFRA, etc).

In general, the inventive polypeptides are detected within a tumor sample that has been obtained from a subject, e.g., a tissue sample, cell sample, cell extract, body fluid sample, etc. The invention encompasses the recognition that the ROR2 polypeptides encoded by the marker genes (or portions thereof) may be present in serum, enabling their detection through a blood test rather than requiring a biopsy specimen. One of ordinary skill in the art will readily be able to develop appropriate assays for polypeptides encoded by the marker genes described herein and to apply them to the detection of such polypeptides in serum. Similar methods may be applied to other body fluid samples, e.g., ascites, urine, saliva, etc.

In certain embodiments, binding can be detected by adding a detectable label to the binding agent. In other embodiments, binding can be detected by using a labeled secondary binding agent that associates specifically with the primary binding agent, e.g., as is well known in the art of antigen/antibody detection. The detectable label may be directly detectable or indirectly detectable, e.g., through combined action with one or more additional members of a signal producing system. Examples of directly detectable labels include radioactive, paramagnetic, fluorescent, light scattering, absorptive and colorimetric labels. Indirectly detectable labels include chemiluminescent labels, e.g., enzymes that are capable of converting a substrate to a chromogenic product such as alkaline phosphatase, horseradish peroxidase and the like.

Once a labeled binding agent has bound a polypeptide marker, the complex may be visualized or detected in a variety of ways, with the particular manner of detection being chosen based on the particular detectable label. Representative detection means include, e.g., scintillation counting, autoradiography, measurement of paramagnetism, fluorescence measurement, light absorption measurement, measurement of light scattering and the like. Depending upon the nature of the sample, appropriate detection techniques include, but are not limited to, immunohistochemistry (IHC), radioimmunoassay, ELISA, immunoblotting and fluorescence activated cell sorting (FACS). In the case where the polypeptide is to be detected in a tissue sample, e.g., a biopsy sample, IHC is a particularly appropriate detection technique.

In general, the detection techniques of the present invention will include a negative control, which can involve applying the test to a control sample (e.g., from a normal tissue) so that the signal obtained thereby can be compared with the signal obtained from the tumor sample being tested. In tests in which a secondary binding agent is used to detect a primary binding agent that binds to the polypeptide of interest, an appropriate negative control can involve performing the test on a portion of the sample with the omission of the primary binding agent.

In general, the results of the inventive detection techniques can be presented in any of a variety of formats. The results can be presented in a qualitative fashion. For example, the test report may indicate only whether or not a particular polypeptide marker was detected, perhaps also with an indication of the limits of detection. The results may be presented in a semi-quantitative fashion. For example, various ranges may be defined, and the ranges may be assigned a score (e.g., 0 to 3 as described in the Examples) that provides a certain degree of quantitative information. Such a score may reflect various factors, e.g., the number of cells in which the polypeptide is detected, the intensity of the signal (which may indicate the level of expression of the polypeptide), etc. The results may be presented in a quantitative fashion, e.g., as a percentage of cells in which the polypeptide is detected, as a protein concentration, etc. As will be appreciated by one of ordinary skill in the art, the type of output provided by a test will vary depending upon the technical limitations of the test and the biological significance associated with detection of the polypeptide. For example, in the case of one polypeptide marker a purely qualitative output (e.g., whether or not the polypeptide is detected at a certain detection level) provides significant information. In another case a more quantitative output (e.g., a ratio of the level of expression of the polypeptide in the sample being tested versus the normal level) is necessary.

In one aspect, the invention provides a method of classifying a GIST or leiomyosarcoma by detecting the presence of ROR2 encoding sequences, e.g. the increased presence of ROR2 mRNA. Generally, the inventive classification methods each include a step of detecting a ROR2 polypeptide, or expression or activity of a gene, including an mRNA, encoding a ROR2 polypeptide. As noted, in certain embodiments it may prove advantageous to combine detection of the ROR2 marker with detection of the KIT and/or PDGFRA markers. The polypeptide or mRNA are detected in a tumor sample.

Although in many cases detection of polypeptides using binding agents such as antibodies represents the most convenient means of determining whether a gene is expressed (or overexpressed) in a particular sample, the invention also encompasses the detection of polynucleotides, e.g., mRNAs for this purpose. Microarray analysis is but one means by which polynucleotides can be used to detect or measure gene expression. Expression of a gene can also be measured by a variety of techniques that make use of a polynucleotide corresponding to part or all of the gene rather than a binding agent for a polypeptide encoded by the gene. Appropriate techniques include, but are not limited to, in situ hybridization, Northern blot, and various nucleic acid amplification techniques such as PCR, quantitative PCR, and the ligase chain reaction. The use of in situ hybridization is described in greater detail in the Examples. PCR and considerations for primer design are well known in the art and are described, for example, in Newton, et al. (eds.) PCR: Essential data Series, John Wiley & Sons; PCR Primer: A Laboratory Manual, Cold Spring Harbor Laboratory Press, Cold Spring Harbor, N.Y., 1995; White, et al. (eds.) PCR Protocols: Current methods and Applications, Methods in Molecular Biology, The Humana Press, Totowa, N.J., 1993.

The invention also encompasses the detection of mutations within a marker gene or within a regulatory region of a marker gene. In certain embodiments of the invention, detection of mutations can be used to further classify a tumor. Mutations may include, but are not limited to, deletions, additions, substitutions, and amplification of regions of genomic DNA that include all or part of a gene. Methods for detecting such mutations are well known in the art and include direct sequencing, denaturing HPLC and combinations thereof. Mutations may result in overexpression or inappropriate expression of the gene. Additionally or alternatively mutations may result in an overly activated gene product (e.g., polypeptide).

It is well known in the art that different tumors subclasses may be associated with different prognoses. Such information may include, but is not limited to, the average life expectancy of a patient, the likelihood that a patient will survive for a given amount of time (e.g., 6 months, 1 year, 5 years, etc.), the likelihood that a patient will be cured of a disease, the likelihood that a patient's disease will respond to a particular therapy (wherein response may be defined in any of a variety of ways). For example, differences in the prognosis of patients with ROR2 positive GIST or leiomyosarcoma are described herein. The present invention therefore offers the possibility of providing diagnostic, prognostic, or predictive information based on the classifying methods. The present invention also offers the possibility of analyzing tumor sample archives containing tissue samples that were obtained from patients and stored with information regarding the progress of the patient's disease. In general such archives consist of tumor samples embedded in paraffin blocks. These tumor samples can be analyzed for their expression of polypeptides encoded by the ROR2 marker genes of the present invention. For example, immunohistochemistry can be performed using antibodies that bind to the polypeptides. Tumors may then be identified on the basis of this information. It is then possible to correlate the classification of a given tumor with available clinical information, e.g., age at death, length of survival, response to therapy, etc. Once suitable prognostic or predictive correlations are identified, a patient's likely outcome can be predicted based on whether his or her tumor belongs to an inventive subclass.

Another aspect of the invention relates to the selection of a treatment regimen based on the inventive classification methods. For example, GLEEVEC® is a tyrosine kinase inhibitor that inhibits KIT but also PDGFRA. Thus, in certain embodiments, the present invention provides a method of classifying a tumor as belonging to a class with a poor prognosis, and then selecting treatment with GLEEVEC® based on the results of that classification step.

It will be appreciated that the inventive methods may be combined with the selection of other known therapies for GIST or leiomyosarcoma. In particular, a number of other therapeutics are currently being developed. For example, SU11248 (manufactured by Pfizer, New York, N.Y.) is a small molecule inhibitor of PDGFRA and KIT that is currently in Phase III clinical trials. This drug is predicted to show utility in treating tumors with KIT or PDGFRA mutations. SU11248 also inhibits VEGFR, thereby providing an additional anti-angiogenic effect. RAD001 (manufactured by Novartis, Switzerland) is currently in Phase I clinical trials. RAD001 inhibits mTOR, a downstream target in the AKT pathway. AKT is a survival pathway that is activated by KIT and many other receptors. It is hoped that the simultaneous inhibition of KIT and mTOR using GLEEVEC® and RAD001 will result in increased effectiveness over GLEEVEC® alone. Novartis have also begun Phase I and II clinical trials with PKC412, an inhibitor of protein kinase C (PKC). PKC412 is less specific than GLEEVEC®, inhibiting PKC, and kinases of KIT, VEGF, PDGF42. Amgen of Thousand Oaks, Calif. are developing AMG706 that is thought to have a similar mechanism of action as SU11248. Bristol-Myers Squibb of New York, N.Y. are developing BMS-354825 that is an inhibitor of both KIT and PDGFRA.

Another aspect of the invention relates to the use of the inventive classification methods in the identification of therapeutics that are subclass specific. Indeed, it is well known in the art that some tumors respond to certain therapies while others do not. The present invention offers the possibility of identifying tumor subclasses characterized by a significant likelihood of response to a given agent, particularly identifying tumors with a high likelihood of metastasis. Tumor sample archives containing tissue samples obtained from patients that have undergone therapy with various agents are available along with information regarding the results of such therapy. As above, these tumor samples can be analyzed for their expression of ROR2 polypeptides. Tumors belonging to different subclasses may then be identified on the basis of this information, and the expression of the ROR2 marker genes correlated with the response of the tumor to therapy, thereby identifying particular compounds that show a superior efficacy in tumors in this subclass as compared with their efficacy in tumors overall or in tumors not falling within that subclass. Once such compounds are identified it will be possible to select patients whose tumors fall into a given subclass for additional clinical trials using these compounds. Such clinical trials, performed on a selected group of patients, are more likely to demonstrate efficacy. The reagents provided herein, therefore, are valuable both for retrospective and prospective trials.

In the case of prospective trials, detection of expression products of one or more of the marker genes may be used to stratify patients prior to their entry into the trial or while they are enrolled in the trial. In clinical research, stratification is the process or result of describing or separating a patient population into more homogeneous subpopulations according to specified criteria. Stratifying patients initially rather than after the trial is frequently preferred, e.g., by regulatory agencies such as the U.S. Food and Drug Administration that may be involved in the approval process for a medication. In some cases stratification may be required by the study design. Various stratification criteria may be employed in conjunction with detection of expression of one or more marker genes. Commonly used criteria include age, family history, lymph node status, tumor size, tumor grade, etc. Other criteria including, but not limited to, tumor aggressiveness, prior therapy received by the patient, etc. Stratification is frequently useful in performing statistical analysis of the results of a trial. Ultimately, once compounds that exhibit superior efficacy against a given GIST or leiomyosarcoma subclass are identified, reagents for detecting expression of the inventive marker genes may be used to guide the selection of appropriate chemotherapeutic agent(s).

In summary, by providing reagents and methods for classifying tumors based on their expression of the marker genes, the present invention offers a means to select suitable therapies. It also offers a means of individualizing therapies to specific subclasses of patients. The invention further provides a means to identify a patient population that may benefit from potentially promising therapies that have been abandoned due to inability to identify the patients who would benefit from their use.

### Therapeutics

The invention encompasses the use of anti-ROR2 antibodies or other antagonists of ROR2 as a therapeutic agent. Such antagonists (which include, but are not limited to, antibodies, small molecules, antisense nucleic acids) may be produced or identified using any of a variety of methods known in the art. For example, a purified polypeptide or fragment thereof may be used to raise antibodies or to screen libraries of compounds to identify those that specifically bind to a ROR2 polypeptide. The fact that ROR2 is a cell membrane associated protein makes it an attractive candidate for antibody therapeutics.

Preferably antibodies suitable for use as therapeutics exhibit high specificity for the target polypeptide and low background binding to other polypeptides. In general, monoclonal antibodies are preferred for therapeutic purposes. Antibodies directed against a polypeptide expressed by a cell may have a number of mechanisms of action. In certain instances, e.g., in the case of a polypeptide that exerts a growth stimulatory effect on a cell, antibodies may directly antagonize the effect of the polypeptide and thereby arrest tumor progression, trigger apoptosis, etc. While not wishing to be bound by any theory, it may be that ROR2 has a growth stimulatory effect on tumor cells or facilitates the growth of such cells in some other way, e.g., by enhancing angiogenesis, by allowing cells to overcome normal growth regulatory mechanisms, or by blocking mechanisms that would normally lead to elimination of mutated or otherwise abnormal cells.

Improved antibodies according to the invention include in particular antibodies with enhanced functional properties. Of special interest are those antibodies with enhanced ability to mediate cellular cytotoxic effector functions such as ADCC. Such antibodies may be obtained by making single or multiple substitutions in the constant framework of the antibody, thus altering its interaction with the Fc receptors. Methods for designing such mutants can be found for example in Lazar et al. (2006, Proc. Natl. Acad. Sci. U.S.A. 103(11): 4005-4010) and Okazaki et al. (2004, J. Mol. Biol. 336(5):1239-49). See also WO 03/074679, WO 2004/029207, WO 2004/099249, WO2006/047350, WO 2006/019447, WO 2006/105338, WO 2007/041635. It is also possible to use cell lines specifically engineered for production of improved antibodies. In particular, these lines have altered regulation of the glycosylation pathway, resulting in antibodies which are poorly fucosylated or even totally defucosylated. Such cell lines and methods for engineering them are disclosed in e.g. Shinkawa et al. (2003, J. Biol. Chem. 278(5): 3466-3473), Ferrara et al. (2006, J. Biol. Chem. 281(8): 5032-5036; 2006, Biotechnol. Bioeng. 93(5): 851-61), EP 1331266, EP 1498490, EP 1498491, EP 1676910, EP 1792987, and WO 99/54342.

In certain embodiments of the invention the antibody may serve to target a toxic moiety to the cell. Thus the invention encompasses the use of antibodies that have been conjugated with a cytotoxic agent, e.g., a toxin such as ricin or diphtheria toxin, a radioactive moiety, etc. Such antibodies can be used to direct the cytotoxic agent specifically to cells that express a ROR2 polypeptide.

Methods are also provided for killing a ROR2⁺ cell by administering to a patient in need thereof an antibody which binds said ROR2 and is able to kill said ROR2⁺ cell by blocking ROR2 biological activity, by inducing apoptosis, ADCC, and/or CDC; or be delivering a cytotoxic moiety. Any of the type of antibodies, antibody fragments, or cytotoxic conjugates as described herein may be used therapeutically. The invention thus includes the use of anti-ROR2 monoclonal antibodies, fragments thereof, or cytotoxic conjugates thereof as medicaments.

Accordingly, the pharmaceutical compositions of the invention are useful in the treatment or prevention of a variety of cancers, including (but not limited to) the following: carcinoma, including that of the bladder, breast, colon, kidney, liver, lung, ovary, pancreas, stomach, cervix, thyroid and skin; including squamous cell carcinoma; hematopoietic tumors of lymphoid lineage, including leukemia, acute lymphocytic leukemia, acute lymphoblastic leukemia, B-cell lymphoma, T-cell lymphoma, Burkitt's lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyoscarama, and osteosarcoma; and other tumors, including melanoma, xeroderma pigmentosum, keratoactanthoma, seminoma, thyroid follicular cancer and teratocarcinoma, and other cancers yet to be determined in which ROR2 is expressed. Cancers of particular interest include breast carcinoma, where it is of particular interest that ROR2 has been found in a sub-population of HER2 negative cells, and thus is of interest as an alternative therapeutic for patients with HER2 negative tumors. Also of interest is the treatment of leiomyosarcoma (LMS) and gastrointestinal stromal tumors (GIST).

The method for inhibiting the growth of selected cell populations can be practiced in vitro, in vivo, or ex vivo. As used herein, “inhibiting growth” means slowing the growth of a cell, decreasing cell viability, causing the death of a cell, lysing a cell and inducing cell death, whether over a short or long period of time.

For clinical in vivo use, the antibody, the epitope-binding antibody fragment, or the cytotoxic conjugate of the invention will be supplied as solutions that are tested for sterility and for endotoxin levels. Examples of suitable protocols of antibody or antibody conjugate administration are as follows: daily, semi-weekly, weekly for at least one week, at least 2 weeks, at least three weeks, at least 4 weeks or more, by any suitable route, e.g. an i.v. bolus each week. Bolus doses may be given in from about 50 to about 100 ml of normal saline to which 5 to 10 ml of human serum albumin can be added. Dosages will vary with the potency of the antibody and the presence of a conjugate, but may be at least 10 μg, at least about 100 μg, at least about 1 mg, at least about 10 mg, at least about 100 mg per administration or more, usually not more than about 1 g per administration, (for example from about 100 ng to about 1 mg/kg per day). Specific clinical protocols with regard to route of administration, excipients, diluents, dosages, times, etc., can be determined by one of ordinary skill in the art as the clinical situation warrants.

Other antagonists of interest function by affecting expression of the polypeptide. Reduction in expression of an endogenously produced polypeptide may be achieved by the administration of antisense nucleic acids (e.g., oligonucleotides, RNA, DNA, most typically oligonucleotides that have been modified to improve stability or targeting) or peptide nucleic acids comprising sequences complementary to those of the mRNA that encodes the polypeptide. Antisense technology and its applications are described in Phillips, M I (ed.) Antisense Technology, Methods Enzymol., Volumes 313 and 314, Academic Press, San Diego, 2000, and references mentioned therein. Ribozymes (catalytic RNA molecules that are capable of cleaving other RNA molecules) represent another approach to reducing gene expression. Such ribozymes can be designed to cleave specific mRNAs corresponding to a gene of interest. Their use is described in U.S. Pat. No. 5,972,621, and references therein. The invention encompasses the delivery of antisense and/or ribozyme molecules via a gene therapy approach in which vectors or cells expressing the antisense molecules are administered to an individual.

Small molecule modulators (e.g., inhibitors or activators) of gene expression are also within the scope of the invention and may be detected by screening libraries of compounds using, for example, cell lines that express a ROR2 polypeptide or a version of a ROR2 polypeptide that has been modified to include a readily detectable moiety. Methods for identifying compounds capable of modulating gene expression are described, for example, in U.S. Pat. No. 5,976,793.

More generally, the invention encompasses compounds that modulate the activity of a marker gene of the present invention. Methods of screening for such interacting compounds are well known in the art and depend, to a certain degree, on the particular properties and activities of the polypeptide encoded by the gene. Representative examples of such screening methods may be found, for example, in U.S. Pat. No. 5,985,829, U.S. Pat. No. 5,726,025, U.S. Pat. No. 5,972,621, and U.S. Pat. No. 6,015,692. The skilled practitioner will readily be able to modify and adapt these methods as appropriate for a given polypeptide. Thus the invention encompasses methods of screening for molecules that modulate the activity of a polypeptide encoded by a marker gene, particularly the ROR2 gene.

The invention also encompasses the use of polynucleotide sequences corresponding to marker genes, or portions thereof, as DNA vaccines. Such vaccines comprise polynucleotide sequences, typically inserted into vectors, that direct the expression of an antigenic polypeptide within the body of the individual being immunized. Details regarding the development of vaccines, including DNA vaccines for various forms of cancer may be found, for example, in Brinckerhoff L H, Thompson L W, Slingluff C L, Melanoma Vaccines, Curr. Opin. Oncol., 12(2):163-73, 2000 and in Stevenson F K, DNA vaccines against cancer: from genes to therapy, Ann. Oncol., 10(12): 1413-8, 1999 and references cited therein. The polypeptides, or fragments thereof, that are encoded by marker genes may also find use as cancer vaccines. Such vaccines may be used for the prevention and/or treatment of cancer.

The invention includes pharmaceutical compositions comprising the antibodies, or small molecule inhibitors, agonists, or antagonists described above. In general, a pharmaceutical composition will include an active agent in addition to one or more inactive agents such as a sterile, biocompatible carrier including, but not limited to, sterile water, saline, buffered saline, or dextrose solution. The pharmaceutical compositions may be administered either alone or in combination with other therapeutic agents including other chemotherapeutic agents, hormones, vaccines, and/or radiation therapy. By “in combination with”, it is not intended to imply that the agents must be administered at the same time or formulated for delivery together, although these methods of delivery are within the scope of the invention. In general, each agent will be administered at a dose and on a time schedule determined for that agent. Additionally, the invention encompasses the delivery of the inventive pharmaceutical compositions in combination with agents that may improve their bioavailability, reduce or modify their metabolism, inhibit their excretion, or modify their distribution within the body. Alternatively or additionally, inventive pharmaceutical compositions may be administered together with one or more other agents that address a symptom or cause of the disease or disorder being treated, or of any other ailment from which the patient suffers. The invention encompasses treating cancer, particularly breast cancer, by administering the pharmaceutical compositions of the invention. Although the pharmaceutical compositions of the present invention can be used for treatment of any subject (e.g., any animal) in need thereof, they are most preferably used in the treatment of humans.

The pharmaceutical compositions of this invention can be administered to humans and other animals by a variety of routes including oral, intravenous, intramuscular, intraarterial, subcutaneous, intraventricular, transdermal, rectal, intravaginal, intraperitoneal, topical (as by powders, ointments, or drops), buccal, or as an oral or nasal spray or aerosol. In general the most appropriate route of administration will depend upon a variety of factors including the nature of the compound (e.g., its stability in the environment of the gastrointestinal tract), the condition of the patient (e.g., whether the patient is able to tolerate oral administration), etc. At present the intravenous route is most commonly used to deliver therapeutic antibodies and nucleic acids. However, the invention encompasses the delivery of the inventive pharmaceutical composition by any appropriate route taking into consideration likely advances in the sciences of drug delivery.

Another aspect of the invention comprises a kit to test for the presence of ROR2 polypeptides or polynucleotides in a tumor sample. The kit can comprise, for example, an antibody for detection of a ROR2 polypeptide or a probe for detection of a polynucleotide. In addition, the kit can comprise a reference or control sample, instructions for processing samples, performing the test and interpreting the results, buffers and other reagents necessary for performing the test. In one embodiment the kit comprises one or more antibodies (monoclonal or polyclonal) for ROR2. In some embodiments monoclonal antibodies are preferred. In other preferred embodiments of the invention, the kit comprises a panel of antibodies or primers, e.g., for ROR2 and KIT; for ROR2 and PDGFRA; or for ROR2, KIT and PDGFRA. In certain embodiments of the invention the kit comprises a cDNA or oligonucleotide array for detecting expression of one or more of the marker genes of the invention. Other kits may include a therapeutic antibody or conjugate thereof and instructions for use.

### EXPERIMENTAL

### Example 1

### ROR2 is a Novel Prognostic Biomarker and a Therapeutic Target in Leiomyosarcoma and Gastrointestinal Stromal Tumour

Described herein are the levels of ROR2 mRNA in 148 soft-tissue sarcomas representing 11 diagnostic subtypes. The expression of ROR2 protein in 573 additional soft-tissue sarcoma samples representing 59 diagnostic subtypes is examined. We also provide evidence that in vitro invasive abilities of LMS and GIST cells are affected by ROR2 expression and that suppression of ROR2 significantly reduces in vivo tumour mass in a xenotransplantation model of LMS. Using tissue microarrays (TMAs) containing tumour samples with known clinical outcome, we further show that high ROR2 expression in LMS and GIST is significantly associated with poor prognosis, and that ROR2 expression is consistent between primary tumours and their metastases. Taken together, these results show that ROR2 is a novel prognostic biomarker and therapeutic target in LMS and GIST.

### Materials and Methods

Case Material.

For gene expression profiling, frozen tissue from 148 soft-tissue tumours was used; this included 61 GIST, 22 LMS, and 12 DTF (Table 51). For confirmation of ROR2 expression by IHC, we used a TMA with 573 cases from 59 sarcoma types (Table 1). For IHC studies on specimens with known clinical outcome data, we studied an additional 410 GIST, 147 LMS, and 90 DTF, which were distributed over 10 TMAs (9-12, Table S3). These tumours were collected from Stanford University Medical Center, the University of Texas M.D. Anderson Cancer Center, and the Cancer Registry of Norway. All cases on the arrays consisted of material obtained at primary diagnosis and had accompanying follow-up data. Only four GIST cases had received imatinib therapy during the period of follow-up. The TMAs were constructed using 0.6 mm cores with a manual tissue arrayer (Beecher Instruments, Silver Spring, Md., USA).

Human Exonic Evidence Based Oligonucleotide (HEEBO) Gene Arrays.

The

HEEBO microarray platform used in the study contained 44,544 70-mer probes that were designed using a transcriptome-based annotation of exonic structure for genomic loci. After confirmation of histology and the presence of viable tumour by frozen section, specimens were homogenized in Trizol reagent (Invitrogen, Carslbad, Calif., USA), and total RNA was extracted. The total RNA was reverse transcribed into cDNA using a mixture of oligo dT (Operon, Huntsville, Ala., USA) and random hexamers (Amersham Biosciences, Little Chalfont Bucks, UK) primers with incorporation of amino allyl-dUTP (Ambion, Austin, Tex., USA). Cy3 and Cy5 dyes (Amersham) were used for indirect labelling of the cDNA from universal human reference RNA (Stratagene, La Jolla, Calif., USA) and cDNA from tumour specimens, respectively. Microarray hybridization and washing was done using standard procedures. Microarrays were scanned on a GenePix 4000 microarray scanner and fluorescence ratios (tumour/reference) were calculated using GenePix software. Only spots with a ratio of signal over a background of at least 1.3 in the Cy5 and 1.5 in the Cy3 channel were included. Gene centering was applied to the expression values for this series of tumours. Only genes with >50% available data were analysed. Data are available for download through the Stanford Microarray Database.

Cell Culture.

LMS04, LMS05, GIST48, and GIST882 cells were derived from primary clinical specimens (LMS04: retroperitoneal lesion that spread from primary uterine LMS tumour; LMS05: primary thigh LMS tumour; GIST48: primary GIST with homozygous exon 11 KIT mutation (V560D) and heterozygous exon 17 KIT mutation (D820A); GIST882: primary GIST with homozygous exon 13 KIT mutation (K642E); ref. 16). LMS04 and LMS05 cells were maintained in RPMI 1640 (Invitrogen) supplemented with 10% foetal bovine serum (FBS, Invitrogen), 100 units/mL penicillin and streptomycin (Invitrogen) and 4 mM L-glutamine (Invitrogen). GIST48 and GIST882 cells were maintained in IMDM (Invitrogen) supplemented with 15% FBS, 100 units/mL penicillin and streptomycin (Invitrogen) and 4 mM L-glutamine (Invitrogen). All cell lines were cultured at 37° C. in 5% CO₂, and the medium was replaced every 2 to 3 days.

Small Interfering RNA Transfections.

LMS04, LMS05, and GIST48 cells were seeded at densities of 8×10⁴ cells per well (in 6-well plates) and 5×10³ cells per well (in 96-well plates) in antibiotic-free medium and allowed to adhere overnight. Cells were transfected with a pool of control siRNAs (siNT, siGENOME Non-Targeting siRNA Pool #1, Dharmacon, Lafayette, Colo., USA) or a pool of siRNAs targeting ROR2 (siROR2, ROR2 siGENOME SMARTpool, Dharmacon). Transfections were carried out with 20 nM siRNA concentrations in OptiMEM (Invitrogen) using Lipofectamine 2000 (Invitrogen) according to the manufacturer's protocol. Efficiencies of siRNA knockdowns were assayed 24 h, 48 h, and 72 h after transfection by quantitative real-time PCR. Cell growth kinetics and cell viability were quantified with a tetrazolium salt (WST-1) colorimetric assay (Roche Molecular Biochemicals, Mannheim, Germany) according to the manufacturer's protocol.

ROR2 Plasmid Transfections.

2×10⁶ GIST882 cells were suspended with 5 μg of purified plasmid DNA in 100 μL of Nucleofector Solution V and electroporated using an Amaxa Nucleofector II machine (program T-030) with full-length human ROR2 or empty control plasmids (OriGene, Rockville, Md., USA). A plasmid encoding TurboGFP (OriGene) was used as a transfection control for all experiments.

Quantitative Real-Time PCR.

RNA was extracted using Trizol (Invitrogen) using standard protocols. Gene expression was quantified using the SYBR green method. Primers were designed to cross intron-exon junctions: ROR2 forward—GGCAGAACCCATCCTCGTG, reverse—CGACTGCGAATCCAGGACC; Wnt5A forward—ACACCTCTTTCCAAACAGGCC, reverse—GGATTGTTAAACTCAACTCTC; β-Actin forward—GCACCCAGCACAATGAAGA, reverse—CGATCCACACGGAGTACTTG. Quantitative real-time PCR was performed on a StepOnePlus instrument (Applied Biosystems, Foster City, Calif.). Transcript levels of target genes were analysed using comparative Cₜ methods, where Cₜ is the cycle threshold number, and normalized to β-Actin.

Matrigel Invasion Assays.

Invasion assays were performed using polyethylene terephthalate invasion chambers with 8.0 μm pores (BD Bioscience, Bedford, Mass., USA). Cells were transfected with siROR2 or siNT for 48 h, serum-starved for 16 h, counted, and seeded (2×10⁵ cells) onto the filters. 20% FBS medium was placed in the lower well to act as a chemoattractant. For Wnt5A treatment, 400 ng/mL of recombinant human Wnt5A (R&D Systems, Minneapolis, Minn.) was added for 16 h prior to invasion. Cells were allowed to invade for 24 h before being fixed in 10% formalin, stained with crystal violet (Sigma-Aldrich, St. Louis, Mo., USA), washed twice with water, and counted. All experiments were performed in triplicate.

Western Blotting and Immunoprecipitation.

Protein lysates were prepared from cell line monolayers using RIPA buffer (Thermo Scientific) supplemented with protease and phosphatase inhibitor cocktails (Roche) and PMSF (Sigma-Aldrich). Protein concentrations were determined with the Bio-Rad Protein Assay (Bio-Rad Laboratories). Immunoprecipitations were performed with an anti-human ROR2 antibody coupled to Protein G Dynabeads (Invitrogen) from 300 μg of total protein according to manufacturer's protocol and 30 μg of whole cell lysate were used as input controls. Denaturing SDS buffer (Invitrogen) was added and samples were heated to 95° C. for 5 minutes. Electrophoresis and immunoblotting were carried out using NuPAGE BisTris gels and nitrocellulose membranes (Invitrogen) according to the manufacturer's protocols. Changes in protein expression and phosphorylation as visualized by chemiluminescence were captured using a GelDoc system (BioRad) and processed using GIMP and lnkscape open-source software. β-Actin was detected using a mouse monoclonal antibody (Sigma-Aldrich), phosphotyrosine was detected using a mouse monoclonal antibody (Cell Signaling Technology), and ROR2 was detected using the monoclonal antibody described previously.

Stable Cell Line Generation.

LMS05 cells were infected with MISSION Lentviral Transduction Particles (Sigma-Aldrich) expressing a non-targeting scramble control shRNA (clone SHC002V) or one of two ROR2-specific shRNAs (ROR2 shRNA #1: clone TRCN0000001492; ROR2 shRNA #2: clone TRCN0000001493). Stable cell lines were selected for with puromycin at a concentration of 2 μg/mL (Sigma-Aldrich).

Xenotransplantation Experiments.

LMS05 cells stably expressing a non-targeting scramble shRNA or one of two ROR2-specific shRNAs were suspended in RPMI containing 25% Matrigel (BD Biosciences) and 100,000 cells were implanted subcutaneously on the backs of 6-week-old NOD/SCID/interleukin (IL)-2γ^{null} (NSG) mice. Animals were euthanized after 8 weeks and tumours were resected, weighed, and fixed in formalin for histologic analysis and IHC. Tumour weights were compared using t-tests. All procedures followed protocols approved by the Stanford Committee on Animal Research.

ROR2 Immunohistochemistry.

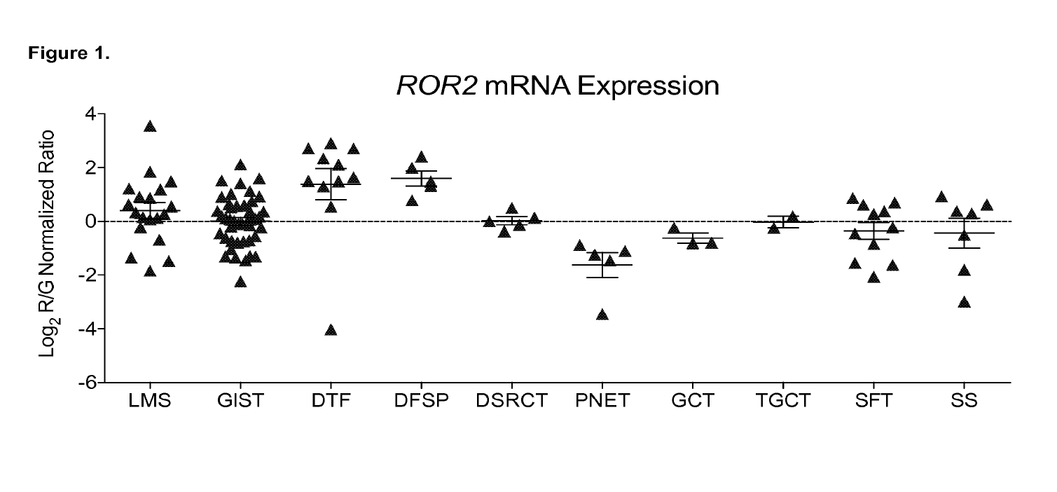

Slides were cut to a thickness of 4 μm, deparaffinized in xylene, and hydrated in a graded series of alcohol. The deparaffinized slides were then boiled by microwave for 12 minutes in citrate buffer (pH 6). A novel primary mouse anti-human ROR2 monoclonal antibody (generated by AM and RN) was used at a 1:25 dilution (13). The IHC reactions were visualized using mouse versions of the EnVision+ system (DAKO, Carpinteria, Calif., USA) using diaminobenzidine. Cores were scored as follows: 2: strong staining whether diffusely or focally present in the tumour; 1: weak staining whether diffusely or focally present in the tumour; 0: absence of any staining (FIG. 1). A score of 2 was considered positive for subsequent statistical analyses.

Statistical Analyses.

Kaplan-Meier analysis in GraphPad Prism V5.0 (GraphPad Software, San Diego, Calif., USA) was used to generate survival curves with log-rank tests to compare patient outcome between groups. Student's t-test was used for comparison of the demographics data wherever appropriate. Multivariate Cox proportional models were generated to assess clinico-pathologic features and their association with patient outcome using the coxph function in the Survival R package. A p-value of less than 0.05 was considered significant.

### Results

ROR2 mRNA and Protein Expression in Soft-Tissue Tumours.

To determine the differences in expression of RTKs in soft-tissue sarcomas, we analysed the mRNA levels of transcripts from 48 different RTKs using gene microarray expression data from 148 soft-tissue tumours. We identified ROR2 as a gene that had low or undetectable levels of expression in the majority of sarcoma subtypes analysed, but that showed high levels of expression in a subset of LMS, GIST, desmoid-type fibromatosis (DTF), and dermatofibrosarcoma protuberans (DFSP) cases (FIG. 1).

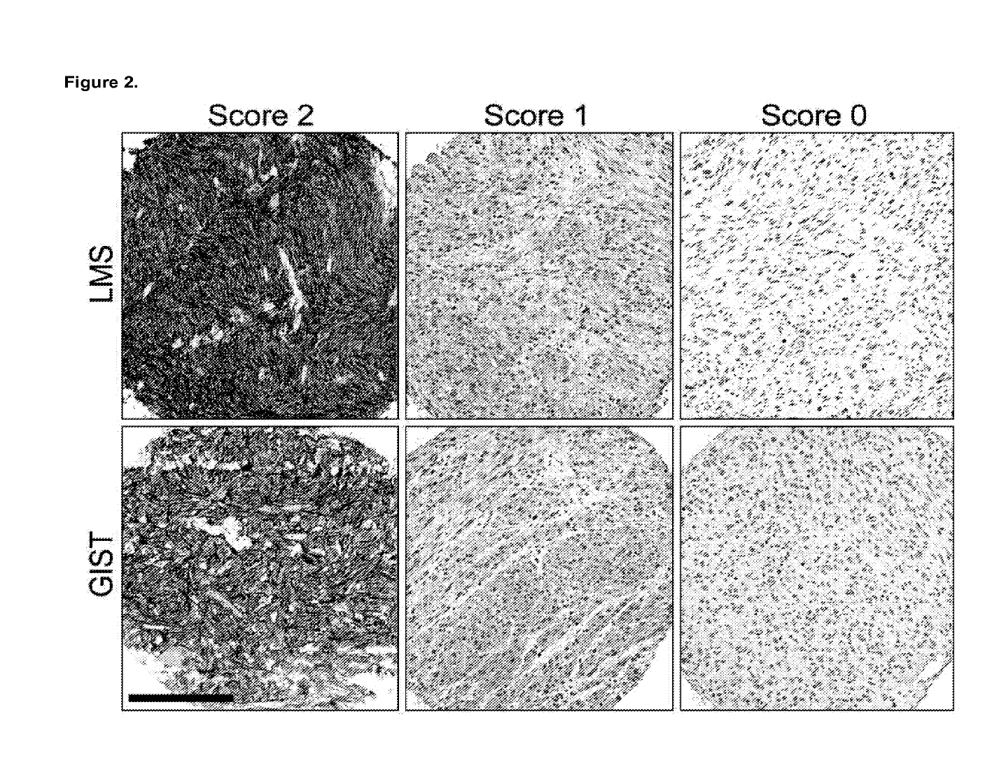

To confirm ROR2 expression in these tumours and to expand on these findings, we next performed an IHC study using TMAs that contained 573 soft-tissue sarcomas and benign soft-tissue tumours; these 573 cases did not overlap with the 148 cases used for gene expression profiling. Similar to our gene array findings, most tumour types on the TMAs failed to react with the ROR2 antibody but a significant subset of LMS, GIST, DTF, and DFSP cases showed strong IHC reactivity (Table 1). Representative stains scored as strongly positive, weakly positive, or negative are shown in FIG. 2 for LMS and GIST. Many tumour samples showed staining of the cytoplasmic membrane, consistent with the predicted localization of ROR2; in some, the membrane staining was obscured by strong staining of the cytoplasm.

ROR2 Expression Mediates the Invasive Abilities of LMS and GIST Cells In Vitro.

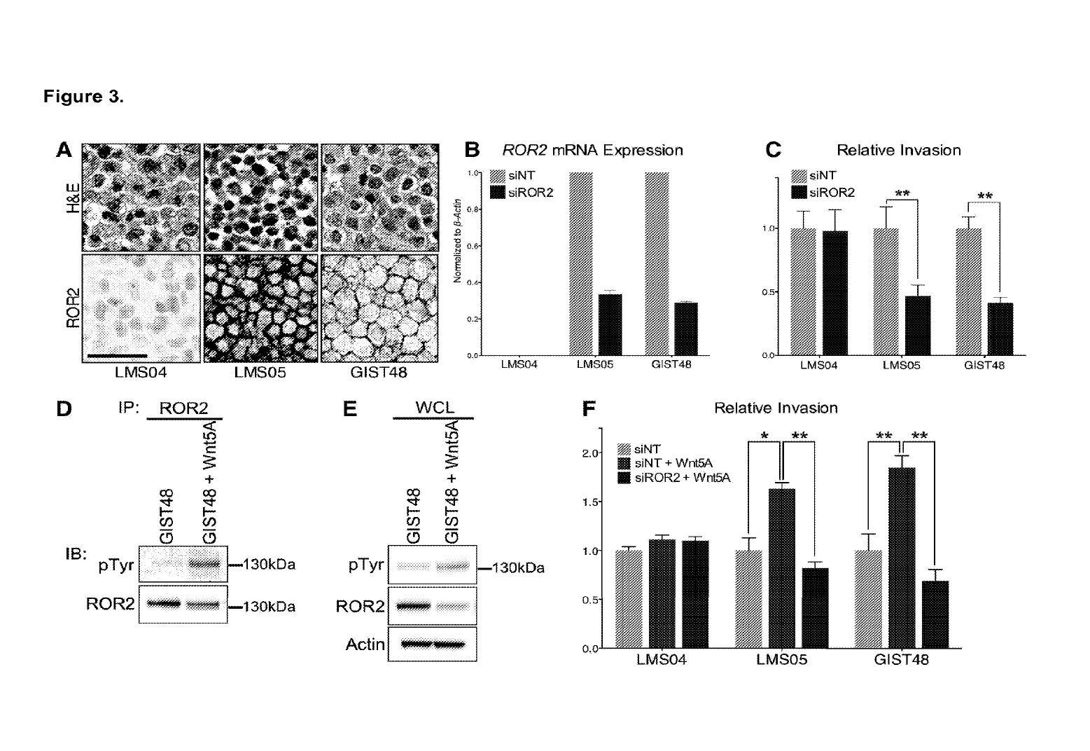

For functional studies, we used two human LMS and two human GIST cell lines. ROR2 mRNA and protein were highly expressed in LMS05 and GIST48, as demonstrated by strong membrane staining for ROR2 protein and high levels of ROR2 transcript; ROR2 expression was undetectable in LMS04 (FIG. 3A). To explore the possibility that ROR2 expression may mediate an aggressive tumour phenotype in soft-tissue sarcomas, we performed in vitro cell proliferation and matrigel invasion experiments using pooled siRNAs targeting ROR2 (siROR2) or non-targeting control siRNAs (siNT) to suppress ROR2. Upon siROR2 treatment, a strong reduction in ROR2 mRNA was observed (FIG. 3B). siRNA treatment did not have an impact on the doubling time or viability of these cell lines. However, in ROR2-positive LMS05 and GIST48 cells, siROR2 treatment led to an approximate 50% reduction in the invasive ability of these cells as compared to siNT treatment; no differences in the invasion rate of ROR2-negative LMS04 were observed (FIG. 3C). Treatment with ROR2-ligand Wnt5A resulted in an increase in endogenous ROR2 receptor activation as measured by tyrosine phosphorylation (FIG. 3D-E) and led to a significant increase in invasion of the ROR2-positive LMS05 and GIST48 cells, an effect which was abrogated by siROR2 treatment. In contrast, ROR2-negative LMS04 showed no significant response to Wnt5A treatment (FIG. 3F).

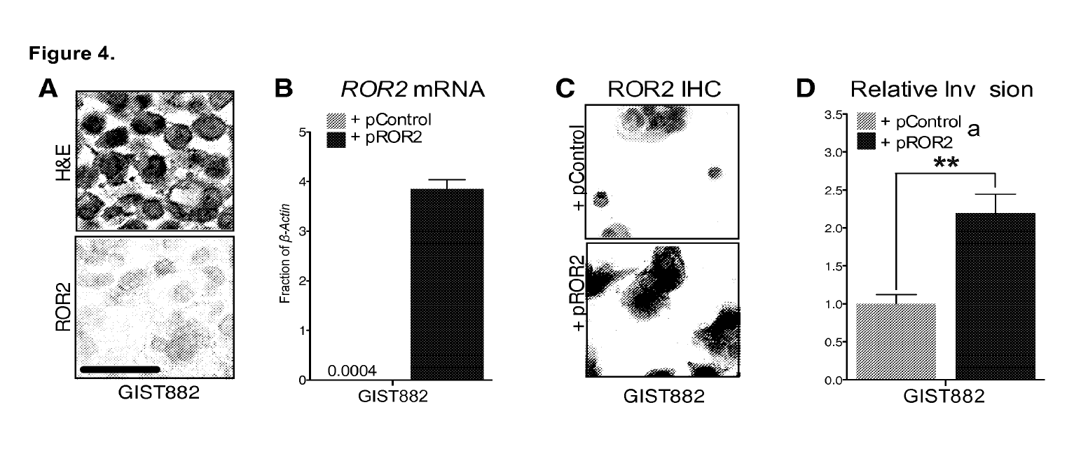

To further investigate whether ROR2 mediates an aggressive sarcoma tumour cell phenotype, we used a fourth sarcoma cell line, GIST882, which expresses very low levels of ROR2 (FIG. 4A). In contrast to ROR2-positive LMS05 and GIST48, and ROR2-negative LMS04, GIST882 had a low baseline rate of in vitro matrigel invasion, thereby making it an ideal candidate to explore the role of ROR2 over-expression in minimally invasive sarcoma cells. Upon transfection with an expression plasmid encoding a full-length human ROR2 cDNA, ROR2 mRNA and protein levels were strongly upregulated (FIG. 4B, C). This ROR2 upregulation had no effect on cell growth kinetics but resulted in a greater than two-fold increase in the in vitro invasion rate of these cells (FIG. 4D). Together, these results demonstrate that ROR2 expression can mediate the invasiveness of soft-tissue sarcoma cells in vitro.

ROR2 Suppression Reduces Tumour Mass In Vivo.

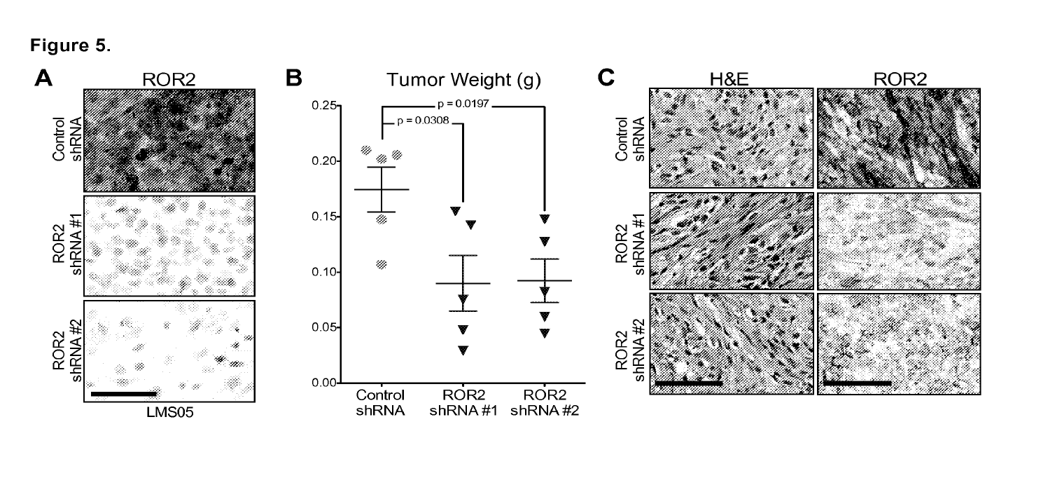

We examined the effects of ROR2 knockdown in vivo by engineering stable ROR2-knockdown cell lines and by developing a xenotransplantation model of LMS. Two ROR2-specific shRNA constructs (shROR2-1 and shROR2-2) and a scrambled shRNA control were stably introduced into ROR2-positive LMS05 cells, and ROR2 knockdown was confirmed (FIG. 5A). Similar to what we observed with transient ROR2 transfections, stable downregulation of ROR2 resulted in no alterations in cell viability or doubling time in vitro. The three cell lines were then engrafted subcutaneously in NSG mice and were allowed to grow for 8 weeks before the mice were euthanized. We found that the mice that had been injected with shROR2-1 and shROR2-2 LMS05 cells showed a 2.6-fold and a 2.9-fold reduction in average tumour mass, respectively, which represented statistically significant decreases as compared to the mice whose LMS05 xenograft tumours expressed the scrambled control shRNA (shROR2-1: P=0.0308; shROR2-2: P=0.0197; FIG. 5B). Histologic examination of the resected xenotransplanted tumours showed that all three LMS05-derivative cell lines maintained similar spindle-cell morphologies in vivo; IHC for ROR2 showed that the downregulation of ROR2 had been maintained in shROR2-1 and shROR2-2 tumours (FIG. 5C).

Prognostic Significance of ROR2 Expression in LMS, GIST, and DTF.

The ROR2-mediated regulation of in vitro tumour invasion in LMS and GIST cells, as well as the reduced tumour growth observed in ROR2-knockdown LMS xenografts, are consistent with the notion that ROR2 may play an important role in soft-tissue sarcoma behaviour. To determine whether ROR2 expression is correlated with patient survival, we performed IHC for ROR2 expression on TMAs with clinical follow-up data. These TMAs were comprised of 147 LMS, 410 GIST, and 90 DTF cases. The clinico-pathologic features of these cases have been published previously and are briefly described below.

The LMS cases consisted of 74 gynecological LMS (Gyn-LMS) and 73 non-gynecological LMS (Non-gyn-LMS); the clinical outcome data available was disease-specific survival (DSS) and the median follow-up time was 3.1 years. None of the LMS patients had received neoadjuvant treatment in the form of chemotherapy and/or radiotherapy. For the 410 GIST cases, overall survival data were available for each patient and the follow-up period was up to 20 years from the time of diagnosis. Only four of the GIST patients analysed had received imatinib treatment during the follow-up period. For the 90 DTF cases, clinical outcome data consisted of time to disease recurrence and the median follow-up period for patients that did not have a recurrence was 5.85 years (range: 0.23 yr to 20.62 yr). For each of the three tumour types, clinical outcome data was used to generate Kaplan-Meier curves and to calculate log-rank (Mantel-Cox) tests to determine whether survival was significantly affected in patients whose tumours expressed ROR2. In addition, a hazard ratio (HR) and its associated 95% confidence interval (CI) were calculated to quantify the effect of ROR2 expression on patient outcomes. For all analyses, cases staining strongly for ROR2 expression (IHC score 2) were compared to those that stained weakly or not at all (IHC scores 1 and 0; FIG. 1).

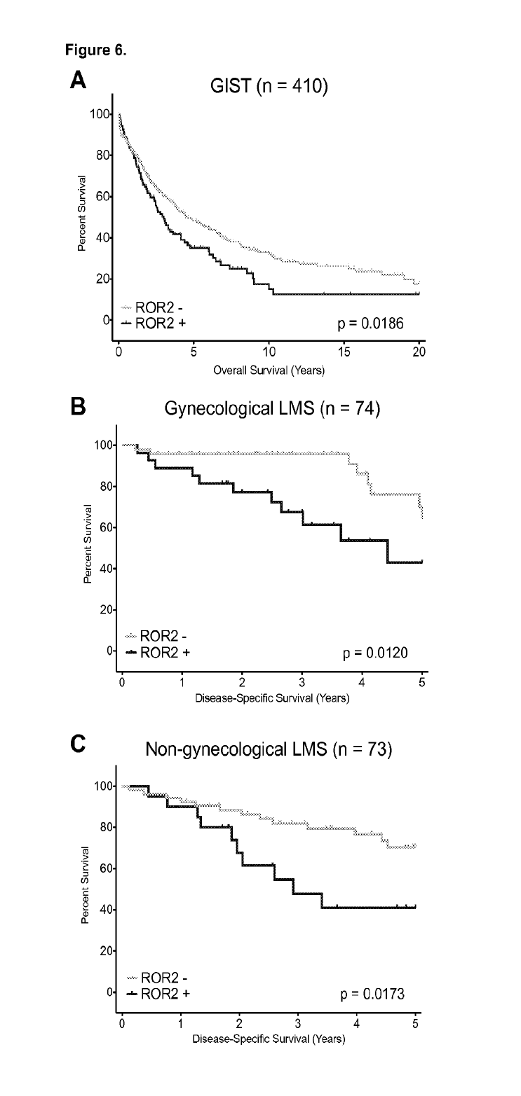

In DTF, we found no significant association between ROR2 expression and disease recurrence (HR=0.9797, CI: 0.5268 to 1.822, P=0.9482). In GIST, tumours with high ROR2 expression were associated with decreased overall survival rates when compared to cases that expressed ROR2 weakly or not at all (HR=1.417, CI: 1.060 to 1.893, P=0.0186; FIG. 6A). LMS patients whose tumour samples were strongly positive for ROR2 had a worse 5-year DSS than those whose tumour samples expressed ROR2 weakly or not at all. This reduction in DSS was seen in equal fashion in both GYN-LMS and ST-LMS (Gyn-LMS: HR=3.497, CI: 1.397 to 9.283, P=0.0120; Non-gyn-LMS: HR=3.287, CI: 1.234 to 8.756, P=0.0173; FIGS. 6B and 6C).

We next assessed whether ROR2 expression was independent of other clinico-pathologic variables in predicting patient survival. For GIST, we constructed multivariate Cox proportional hazards models that considered ROR2 expression, tumor size, mitotic rate, and anatomical location. For gynecological and non-gynecological LMS, similar multivariate analyses were conducted using ROR2 expression, Ki67 status, Federation Nationale des Centres de Lutte Contre le Cancer (FNLCC) grade, and necrosis as inputs. Despite its prognostic utility as a stand-alone IHC marker, we found that high ROR2 expression was not associated with survival in GIST or LMS independent of the other clinico-pathologic features considered.

ROR2 Expression is a Stable Property of LMS Tumours.

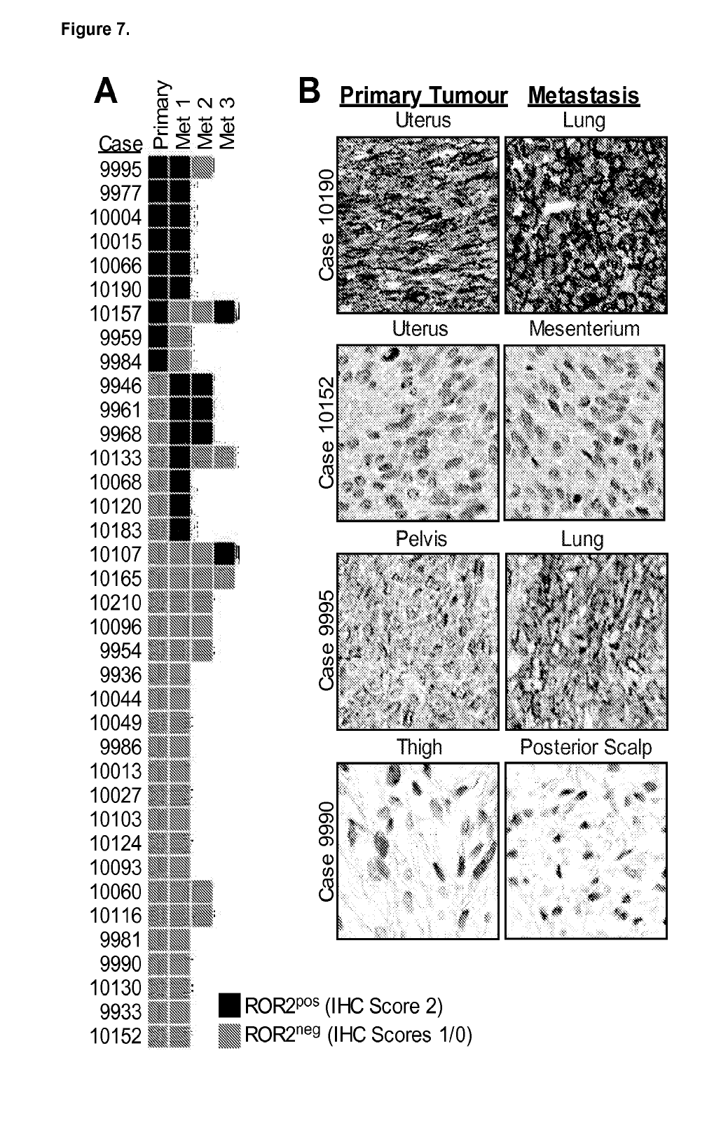

We next examined the consistency of ROR2 expression between 37 primary gynecological and non-gynecological LMS samples and their corresponding metastatic lesions. We found that in primary tumours that were scored as ROR2-positive by IHC (Score: 2, FIG. 2), 7 of 9 (77.8%) maintained high ROR2 expression in at least one of their distant metastases. In primary tumours with low or absent ROR2 expression (Score: 1 or 0, FIG. 2), 20 of 28 (71.4%) maintained this lack of ROR2 expression in their associated metastases whereas 8 of 28 (28.6%) of tumours showed a gain of strong ROR2 expression in at least one of their metastatic growths (FIG. 7).

### Discussion

Experimental and clinical studies have shown that deregulated RTKs can play important roles in cancer development and progression. Furthermore, RTKs have proven to be amenable therapeutic targets as is evidenced by several FDA-approved antibody and small molecule drugs targeting RTKs; these therapeutics have showed clinical efficacy in a wide range of cancer types.

Here we present RTK gene expression data in 148 soft-tissue sarcomas. One of these RTKs, ROR2, showed significant variability in expression in LMS, GIST and DTF. ROR2 is known to regulate cell migration during vertebrate development by acting as a receptor or co-receptor for Wnt5A. Recently, in vitro and xenograft experiments have shown that the Wnt5A-ROR2 signalling cascade is important for the invasive abilities of melanoma, osteosarcoma, and RCC cell lines, thereby making ROR2 a candidate biomarker of tumours with aggressive growth potential or as a therapeutic target.

In the current study, we present the first large-scale characterization of ROR2 expression in human soft-tissue sarcomas. The initial gene expression results were confirmed and expanded to a larger number of sarcomas on 573 cases representing 59 tumour types by IHC on TMAs. In addition to the tumour types identified by gene expression profiling, small numbers of ROR2-positive cases were found in high grade undifferentiated sarcoma and a significant subset of dermatofibrosarcoma protuberans. In a significant proportion of cases strong levels of expression at the cell membrane was found. Similar to the immunohistochemical scoring used for HER2 in breast cancer, only those cases that showed strong membrane reactivity were scored as positive.

Similar to results shown for melanoma, osteosarcoma, and RCC cell lines, inhibition of ROR2 expression strongly decreased the in vitro invasiveness of two highly invasive ROR2-positive LMS and GIST cell lines. A third invasive cell line (LMS04), derived from an ROR2-negative LMS, also showed significant invasive capacity in vitro and exhibited no difference in invasion under the same experimental conditions, indicating that while ROR2 is functionally important for the subset of tumours in which it is expressed it is not the only factor that determines tumour cell invasiveness. Treatment of ROR2-positive GIST48 cells with ROR2-specific ligand Wnt5A increased the activation of endogenous ROR2, as measured by immunoblotting for phosphotyrosine in cell lysates precipitated with an anti-ROR2 antibody. Previous studies highlighting the interaction between ROR2 and Wnt5A in human tumour cell lines relied on experiments utilizing exogenously expressed ROR2 cDNA constructs; our data, in contrast, show that Wnt5A indeed activates endogenous ROR2. Concomitant with this increase in ROR2 receptor activation was a significant increase in cell invasion of ROR2-positive GIST48 and LMS05; upon ROR2 down-regulation, however, this increased invasion was strongly abrogated. This suggests that in ROR2-positive LMS and GIST cells, the aggressive tumour phenotype induced by non-canonical Wnt signalling is likely mediated through ROR2. A fourth cell line (GIST882), derived from an ROR2-negative, minimally invasive GIST tumour, demonstrated a marked increase in its in vitro invasive capacity upon transfection with a plasmid encoding full-length human ROR2 as compared to a control plasmid, further demonstrating a potential role for ROR2 in mediating an aggressive tumour phenotype.

In addition, two independent ROR2 knockdown LMS cell lines derived from LMS05 showed diminished in vivo growth capacity as compared to an isogenic control cell line in a xenotransplantation model of LMS. While decreasing ROR2 expression did not diminish cell proliferation in vitro, the smaller tumour sizes observed in ROR2-down-regulated xenotransplanted tumours may result from an impairment of these cells to successfully invade into surrounding tissues, thereby decreasing the exposure of cell-surface growth factors to proliferative signals being secreted by normal cells in the tumour microenvironment.

The ROR2-mediated effects seen on cell invasion and xenotransplanted tumour growth suggest a clinically significant role for the ROR2 molecule. No other reports on the clinical significance of ROR2 expression have previously been made in the literature. To determine the possible role for ROR2 in the clinical behaviour of tumours, we studied ROR2 expression on TMAs containing LMS and GIST cases with known clinical follow-up. Here, we provide the first prognostic association of ROR2 protein expression with poor clinical outcome in cancer. In both LMS and GIST, ROR2 expression is associated with poor clinical outcome, demonstrating that ROR2 plays an important role in the behaviour of these tumors and indicating that ROR2 is an amenable therapeutic target in these tumor types. When evaluating a novel therapeutic target it is also important to determine the stability of expression of the marker. Here we show that the high ROR2 expression in primary LMS tumors is maintained in the majority (approx. 80%) of metastases. Furthermore, in primary tumors with low or absent ROR2 expression, nearly 30% saw a gain in ROR2 expression in their associated metastases, thereby suggesting that an anti-ROR2 therapy can be efficacious in both primary and secondary tumours.

Currently, there exist no targeted therapies for LMS, thereby making ROR2 an attractive therapeutic target given the prognostic associations and experimental findings reported herein. The majority of GIST tumours show activation of the tyrosine kinase proteins KIT or PDGFRA and specific mutations in the genes transcribing these proteins predict response to the tyrosine kinase inhibitor imatinib and other small molecule therapies. However, almost all GIST patients eventually develop resistance to treatment, thereby necessitating the exploration of other therapeutic targets, such as ROR2. The data currently available for ROR2 share some important similarities with those for HER2 expression in breast cancer. For both RTKs, a subset of patients show strong expression of the molecule at the cell surface and in both cases, this expression is associated with poor clinical behaviour. HER2-positive breast cancers have, in the majority of cases, an amplification of the HER2 gene; future studies will need to be performed for ROR2 to determine whether a similar mechanism occurs in LMS and GIST tumours. However, gene amplification is not the only mechanism through which RTKs can be involved in tumorigenesis.

In summary, ROR2 is highly expressed in a subset of LMS, GIST, and DTF cases and high ROR2 protein expression is significantly associated with poor clinical outcome in patients with LMS and GIST. ROR2 expression is maintained in distant metastases of LMS tumours, and in some cases is re-activated in these secondary lesions compared to the primary tumours from which they originated, thereby highlighting its stability as a therapeutic target in these cancers. Further, ROR2 expression mediates the in vitro invasive abilities of LMS and GIST cells and significantly diminishes in vivo tumour mass when down-regulated in a xenotransplantation model of LMS. Wnt5A, a known ligand for ROR2, increases activation of endogenously expressed ROR2 in tumour cells and increases the in vitro invasiveness of these cells through matrigel. Taken together, these results not only demonstrate the utility of ROR2 as a prognostic biomarker, but also that ROR2 represents a novel therapeutic target for the treatment of GIST and LMS.

### LIST OF ABBREVIATIONS

### DFSP Dermatofibrosarcoma Protuberans

### DSRCT Desmoplastic Small Round Cell Tumour

### DTF Desmoid-type Fibromatosis

### GCT Giant Cell Tumour

### GIST Gastrointestinal Stromal Tumour

### HEEBO Human Exonic Evidence Based Oligonucleotide

### HR Hazard Ratio

### IHC Immunohistochemistry

### LMS Leiomyosarcoma

### PNET Primitive Neuroectodermal Tumour

### RCC Renal Cell Carcinoma

### ROR2 Receptor Tyrosine Kinase-Like Orphan Receptor 2

### RTK Receptor Tyrosine Kinase

### SFT Solitary Fibrous Tumour

shRNA Short Hairpin RNA
siRNA Small Interfering RNA

### SS Synovial Sarcoma

### TGCT Tenosynovial Giant Cell Tumour

### TMA Tissue Microarray

TABLE 1
ROR2 IHC Staining Results in Soft-Tissue Sarcomas
and Benign Soft-Tissue Tumours

| | Negative | Weak | Strong | Total |
|---|---|---|---|---|
| Leiomyosarcoma | 34 | 12 | 14 | 60 |
| Gastrointestinal stromal tumour | 20 | 23 | 7 | 52 |
| Fibromatosis | 14 | 11 | 1 | 26 |
| Pleomorphic sarcoma | 50 | 11 | 4 | 65 |
| Tenosynovial giant cell tumour | 26 | 0 | 0 | 26 |
| Liposarcoma | 18 | 4 | 2 | 24 |
| Leiomyoma | 20 | 1 | 0 | 21 |
| Schwanoma | 20 | 1 | 0 | 21 |
| Solitary fibrous tumour | 14 | 5 | 0 | 19 |
| Synovial sarcoma | 9 | 7 | 2 | 18 |
| Rhabdomyosarcoma | 13 | 0 | 0 | 13 |
| Endometrial stroma sarcoma | 7 | 3 | 2 | 12 |
| Neurofibroma | 12 | 0 | 0 | 12 |
| Epithelioid hemangioendothelioma | 9 | 2 | 0 | 11 |
| Extraskeletal myxoid | 9 | 0 | 1 | 10 |
| chondrosarcoma | | | | |
| Angiosarcoma | 9 | 1 | 0 | 10 |
| Fibroadenoma | 8 | 2 | 0 | 10 |
| Dermatofibrosarcoma protuberans | 2 | 3 | 4 | 9 |
| Osteosarcoma | 4 | 2 | 3 | 9 |
| Angiomyolipoma | 2 | 3 | 3 | 8 |
| Carcino-sarcoma | 4 | 2 | 2 | 8 |
| Pigmented villonodular synovitis | 8 | 0 | 0 | 8 |
| Fibroma of ovary | 7 | 0 | 0 | 7 |
| Ewing sarcoma | 7 | 0 | 0 | 7 |
| Giant cell tumour of the bone | 5 | 2 | 0 | 7 |
| Non-ossifying fibroma | 6 | 1 | 0 | 7 |
| Malignant peripheral nerve sheath | 4 | 1 | 1 | 6 |
| tumours | | | | |
| Nodular fascitis | 5 | 1 | 0 | 6 |
| Fibroma of tendon sheath | 4 | 2 | 0 | 6 |
| Glomus tumour | 4 | 2 | 0 | 6 |
| Bone fibrous dysplasia | 3 | 1 | 1 | 5 |
| Fibroxanthoma | 3 | 2 | 0 | 5 |
| Myxoid fibrosarcoma | 5 | 0 | 0 | 5 |
| Inflammatory myofibroblastic tumour | 5 | 0 | 0 | 5 |
| Desmoplastic small round cell tumour | 1 | 2 | 1 | 4 |
| Granular cell tumour | 4 | 0 | 0 | 4 |
| Myxoma | 4 | 0 | 0 | 4 |
| Neuroblastoma | 2 | 0 | 1 | 3 |
| Epithelioid sarcoma | 3 | 0 | 0 | 3 |
| Low grade fibromyxoid sarcoma | 3 | 0 | 0 | 3 |
| Hemagiopericytoma | 2 | 0 | 0 | 2 |
| Kaposi sarcoma | 2 | 0 | 0 | 2 |
| Lymphoangioma vascular | 2 | 0 | 0 | 2 |
| Clear cell sarcoma | 2 | 0 | 0 | 2 |
| Chondromyxoid fibroma | 2 | 0 | 0 | 2 |
| Fibrosarcoma | 2 | 0 | 0 | 2 |
| Adamantinoma | 1 | 1 | 0 | 2 |
| Chondrosarcoma | 1 | 1 | 0 | 2 |
| Phyllodes tumour | 2 | 0 | 0 | 2 |
| Embryonal sarcoma | 0 | 0 | 1 | 1 |
| Aggresive angiomyxoma | 1 | 0 | 0 | 1 |
| Alveolar soft part sarcoma | 0 | 1 | 0 | 1 |
| Hemangioma | 1 | 0 | 0 | 1 |
| Juvenile xanthogranuloma | 1 | 0 | 0 | 1 |
| Primitive neuroectodermal tumour | 1 | 0 | 0 | 1 |
| Atypical lipomatous tumour | 1 | 0 | 0 | 1 |
| Aneurysmal bone cyst | 0 | 1 | 0 | 1 |
| Enchondroma | 1 | 0 | 0 | 1 |
| Fibrohistiocytoma of bone | 1 | 0 | 0 | 1 |

### Example 2

### Binding of an Anti-ROR2 Monoclonal Antibody to Live Cancer Cells

ROR2-negative LMS04 cells and ROR2-positive LMS05 and GIST48 cells were dissociated with TrypLE (Life Technologies), quenched with growth medium (Invitrogen), passed through a 70-micron filter (BD Biosciences), spun down, and resuspended at a concentration of 1×10⁶ cells/mL in MACS Buffer (Miltenyi Biotec). The cells were then Fc-blocked for 10 minutes by the addition of 100 ug/mL mouse IgG before being incubated with 1 μg anti-ROR2 monoclonal antibody (R&D Systems Human ROR2 Alexa Fluor 488 MAb, Clone 231509, Mouse IgG2A) or isotype control antibody (R&D Systems) for 30 minutes at 4 C. The cells were then washed twice in MACS buffer, stained with DAPI, and analyzed for cell-surface ROR2 expression on an LSRFortessa cell analyzer (BD Biosciences). The experiment was performed in biological duplicates and least 10,000 events were counted for each experimental replicate. Consistent with immunohistochemical and qRT-PCR analysis, shown in FIG. 8, ROR2 expression was not detected on the surface of live LMS04 cells (A) but was detected on ROR2-positive LMS05 (B) and GIST48 (C). The area shaded in red and outlined in black represents cells stained with the ROR2 mAb, while the area without shading and outlined in black represents cells stained with an isotype control antibody

### Example 3

### Evaluation of ROR2 Expression in Normal and Cancer Tissues by Immunohistochemistry

Slides were cut to a thickness of 4 μm, deparaffinized in xylene, and hydrated in a graded series of alcohol. The deparaffinized slides were then boiled by microwave for 12 minutes in citrate buffer (pH 6). A novel primary mouse anti-human ROR2 monoclonal antibody was used at a 1:25 dilution. The IHC reactions were visualized using mouse versions of the EnVision+ system (DAKO, Carpinteria, Calif., USA) using diaminobenzidine. Cores were scored as follows: 2: strong staining whether diffusely or focally present in the tumour; 1: weak staining whether diffusely or focally present in the tumour; 0: absence of any staining. A score of 2 was considered positive for statistical analyses in data sets were patient clinical outcome information were available.

In all, we evaluated the following tissue samples immunohistochemistry: leiomyosarcoma tissue microarray (TMA) with clinical follow-up data (n=147), gastrointestinal stromal tumor TMA with clinical follow-up data (n=414), desmoid-type fibromatosis TMA with clinical follow-up data (n=90), breast carcinoma TMA (n=281), ductal carcinoma in situ TMA (n=284), invasive ductal carcinoma TMA (n=136), normal tissue samples (n=136), endometrial carcinoma TMA (n=221,), tuberous sclerosis-associated tumor samples (n=57), renal cell carcinoma samples (n=211), Wilm's tumor samples (n=13), adrenal neuroblastoma samples (n=27), pan-sarcoma and benign soft-tissue tumor TMA (n=573), pan-carcinoma TMA (n=368), and osteosarcoma TMA (n=83).

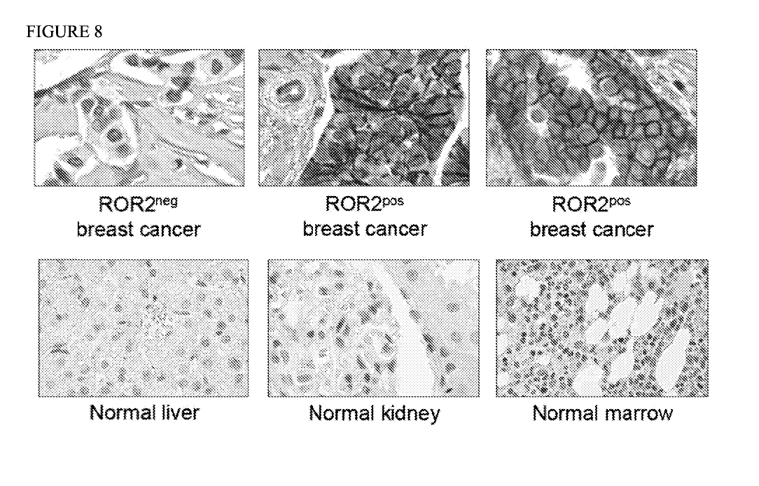

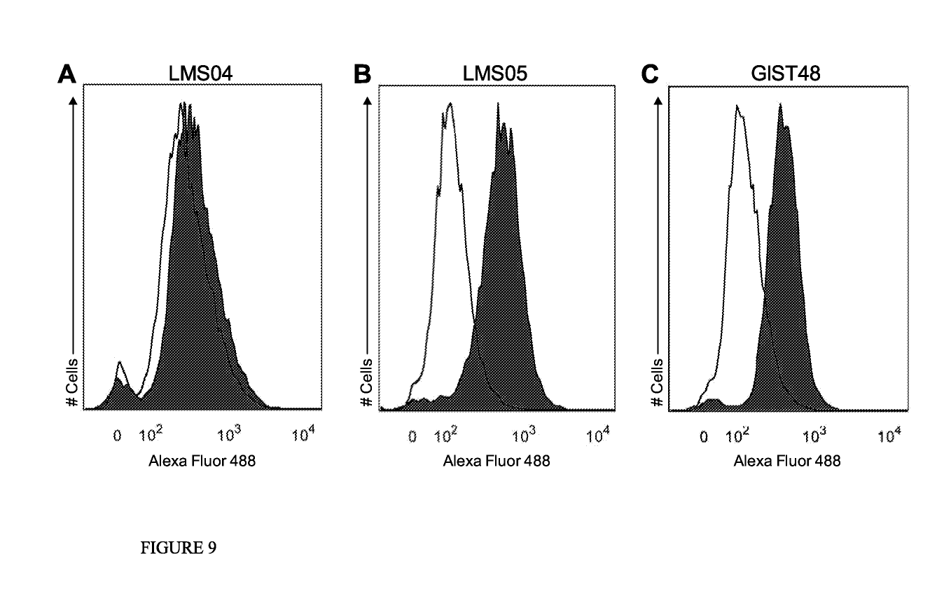

The results from breast cancer samples are shown in Table 2, and in FIG. 9. For breast cancer, there was no overlap between HER2 expression and ROR2 expression. The numbersrepresent different assignments of staining grade: 3=strong, 2/1=weak, 0=absent, missing=data could not be evaluated (i.e., the tissue core came off that particular slide of the tissue microarray, so that case could not be evaluated).

| | | | | | | ROR2 - | |
|---|---|---|---|---|---|---|---|
| ER | HER2 | ROR2-0 | ROR2-1 | ROR2-2 | ROR2-3 | missing | N |
| ER: equivocal | HER2-NA | 1 | | | | | 1 |
| ER-NA | HER2-NA | 9 | | | | 1 | 10 |
| ER: neg | HER2-NA | 1 | | | | | 1 |
| | HER2: neg | 21 | 2 | 2 | 3 | 3 | 31 |
| | HER2: equivocal | | | | | | |
| | (1.8-2.2) | | 1 | | | | 1 |
| | HER2: pos | 6 | | | | 2 | 8 |
| ER: pos | HER2-NA | 9 | 3 | 1 | 2 | 1 | 16 |
| | HER2: neg | 114 | 14 | 9 | 8 | 16 | 161 |
| | HER2: equivocal | | | | | | |
| | (1.8-2.2) | 7 | 1 | | | | 8 |
| | HER2: pos | 7 | | 2 | | 2 | 11 |
| Carcinoma. | | | | | | | |
| NoER. | | | | | | | |
| HER2.infor | | 20 | 2 | 2 | 2 | 7 | 33 |
| Total | | 195 | 23 | 16 | 15 | 32 | 281 |

| | | | | | ROR2- | |
|---|---|---|---|---|---|---|
| Diagnosis | ROR2-0 | ROR2-1 | ROR2-2 | ROR2-3 | missing | N |
| DCIS | 9 | 2 | 3 | | | 14 |
| DCIS alone | 78 | 21 | 15 | 4 | 5 | 123 |
| DCIS only | 13 | | | | 2 | 15 |
| DCIS with focal | | | 1 | | | 1 |
| IDC | | | | | | |
| DCIS with focal | 1 | | | | | 1 |
| ILC | | | | | | |
| DCIS with IDC | 64 | 16 | 12 | 1 | 3 | 96 |
| DCIS with | | | | | | |
| invasive | 13 | 6 | 7 | 6 | 2 | 34 |
| Total | 178 | 45 | 38 | 11 | 12 | 284 |

| | | | | | ROR2- | |
|---|---|---|---|---|---|---|
| Diagnosis | ROR2-0 | ROR2-1 | ROR2-2 | ROR2-3 | missing | N |
| Invasive Ductal | 111 | 12 | 6 | 4 | 3 | 136 |
| Carcinoma | | | | | | |

TABLE 3
Renal and Adrenal Cancer

| | Negative | Moderate/Strong | N |
|---|---|---|---|
| Renal Cell Carcinoma - Clear Cell | 152 | 16 | 168 |
| Renal Cell Carcinoma - | 42 | 1 | 43 |
| Transitional Cell | | | |
| Wilm's Tumor | 1 | 12 | 13 |
| Adrenal Neuroblastoma | 22 | 5 | 27 |

TABLE 4
Pan Carcinoma

| | Negative | Weak | Strong | N |
|---|---|---|---|---|
| Breast Carcinoma | | | | |
| Lobular | 7 | 0 | 0 | 7 |
| Metaplastic | 1 | 1 | 0 | 2 |
| Mixed | 1 | 0 | 0 | 1 |
| Ductal | 4 | 3* | 0 | 7 |
| NOS | 1 | 0 | 0 | 1 |
| Esophagus Carcinoma | | | | |
| SCC | 3 | 1 | 0 | 4 |
| Adeno | 4 | 0 | 0 | 0 |
| Spindle cell | 3 | 0 | 0 | 0 |
| Liver | | | | |
| HCC | 7 | 0 | 0 | 7 |
| Adenoma | 2 | 0 | 0 | 2 |
| Hemangioendothelioma epithelioid | 1 | 0 | 0 | 1 |
| Hemangioendothelioma infantile | 1 | 0 | 0 | 1 |
| Hemangioma | 2 | 0 | 0 | 2 |
| Stomach Carcinoma | | | | |
| Adeno | 8 | 1* | 0 | 9 |
| Pancreas | | | | |
| Adenocarcinoma | 6 | 0 | 0 | 6 |
| Endocrine tumor | 3 | 1 | 0 | 4 |
| Mucinous cystic tumor | 4 | 0 | 0 | 4 |
| Papillary cystic tumor | 2 | 0 | 0 | 2 |
| Serous microcystic adenoma | 1 | 1* | 0 | 2 |
| Adrenal gland | | | | |
| Neuroblastoma | 0 | 1 | 0 | 1 |
| Cortical adenoma | 3 | 0 | 0 | 3 |
| Cortical carcinoma | 2 | 0 | 0 | 2 |
| Pheochromocytoma | 0 | 2 | 0 | 2 |
| Bile duct | | | | |
| Cholangiocarcinoma | 4 | 0 | 0 | 4 |
| Biliary cystadenoma | 2 | 0 | 0 | 2 |
| Hamartoma | 2 | 0 | 0 | 2 |
| Kidney | | | | |
| Clear cell carcinoma | 5 | 0 | 1 | 6 |
| Papillary renal cell carcinoma | 1 | 0 | 0 | 1 |
| Chromophobe carcinoma | 3 | 0 | 0 | 3 |
| Oncocytoma | 2 | 0 | 0 | 2 |
| Transitional cell carcinoma from the | 4 | 0 | 0 | 4 |
| renal pelvis | | | | |
| Angiomyoma | 0 | 1 | 0 | 1 |
| Neuroblastoma | 0 | 0 | 2 | 2 |
| Mesoblastic nephroma | 0 | 1 | 0 | 1 |
| Colon | | | | |
| Adenocarcinoma | 5 | 4* | 0 | 9 |
| Adenoma | 3 | 0 | 0 | 3 |
| Appendix | | | | |
| Carcinoid tumor | 2 | 1 | 0 | 3 |
| Ovary | | | | |
| Mucinous borderline tumor | 1 | 0 | 0 | 1 |
| Serous borderline tumor | 3 | 0 | 0 | 3 |
| Endometrioid carcinoma | 0 | 0 | 1 | 1 |
| Mucinous carcinoma | 2 | 0 | 0 | 2 |
| Serous carcinoma | 1 | 0 | 0 | 1 |
| Clear cell carcinoma | 4 | 0 | 0 | 4 |
| Malignant mixed Mullerian tumor | 0 | 0 | 2 | 2 |
| Dysgerminoma | 0 | 0 | 1 | 1 |
| Fibroma | 1 | 0 | 0 | 1 |
| Granulosa cell tumor, juvenile | 1 | 0 | 0 | 1 |
| Granulosa cell tumor, adult | | | | |
| Sertoli Leydig cell tumor | 1 | 1 | 0 | 2 |
| Steroid cell tumor | 1 | 0 | 0 | 1 |
| Teratoma, mature | 0 | 0 | 1 | 1 |
| Yolk sac tumor | 0 | 0 | 1 | 1 |
| Peritoneum | | | | |
| Serous carcinoma | 1 | 0 | 0 | 1 |
| Urinary bladder | | | | |
| Transitional cell carcinoma | 9 | 2* | 0 | 11 |
| Adenocarcinoma | 2 | 1* | 0 | 3 |
| Cloacogenic carcinoma | 1 | 0 | 0 | 1 |
| Small cell carcinoma | 1 | 0 | 0 | 1 |
| Squamous cell carcinoma | 1 | 0 | 0 | 1 |
| Undifferentiated carcinoma | 1 | 0 | 0 | 1 |
| Papilloma | 0 | 1* | 0 | 1 |
| Uterus | | | | |
| Leiomyoma | 1 | 0 | 0 | 1 |
| Leiomyosarcoma | 1 | 2 | 1 | 4 |
| Malignant mixed Mullerian tumor | 0 | 0 | 1 | 1 |
| Endometrial carcinoma | 6 | 2 | 1 | 9 |
| Papillary serous carcinoma | 1 | 0 | 1 | 2 |
| Endometrial stromal sarcoma | 1 | 1 | 1 | 3 |
| Prostate | | | | |
| Adenocarcinoma | 8 | 2 | 0 | 10 |
| Clear cell carcinoma | 3 | 0 | 0 | 3 |
| Transitional cell carcinoma | 1 | 0 | 0 | 1 |
| Squamous cell carcinoma | 1 | 0 | 0 | 1 |
| Prostatic intraepithelial neoplasia | 0 | 1 | 0 | 1 |
| Uterine cervix | | | | |
| Squamous cell carcinoma | 4 | 1 | 0 | 5 |
| Adenocarcinoma | 2 | 2 | 0 | 4 |
| Vulva | | | | |
| Paget's disease | 1 | 0 | 0 | 1 |
| Squamous cell carcinoma | 0 | 1 | 0 | 1 |
| Testis | | | | |
| Seminoma | 10 | 10 | 1 | 21 |
| Embryonal carcinoma | 3 | 0 | 0 | 3 |
| Yolk sac tumor | 0 | 1 | 1 | 2 |
| Teratoma | 1 | 1 | 0 | 2 |
| Mixed teratoma and embryonal | 1 | 0 | 0 | 1 |
| carcinoma | | | | |
| Mixed germ cell tumor | 4 | 1 | 1 | 6 |
| Leydig cell tumor | 1 | 0 | 0 | 1 |
| Granulosa cell tumor, juvenile | 1 | 0 | 0 | 1 |
| Adenomatoid tumor | | | | |
| Fibrous pseudotumor | 1 | 0 | 0 | 1 |
| Skin | | | | |
| Basal cell carcinoma | 1 | 0 | 0 | 1 |
| Squamous cell carcinoma | 2 | 1* | 0 | 3 |
| Merkel cell carcinoma | 2 | 1 | 0 | 3 |
| Adenocarcinoma | 1 | 0 | 0 | 1 |
| Melanoma (no desmoplastic) | 20 | 1 | 0 | 21 |
| Melanoma (desmoplastic) | 1 | 0 | 0 | 1 |
| Oral cavity | | | | |
| Squamous cell carcinoma | 4 | 0 | 0 | 4 |
| Squamous papilloma | 2 | 0 | 0 | 2 |
| Brain | | | | |
| Pilocytic astrocytoma | 2 | 0 | 0 | 2 |
| Oligodentroglioma | 2 | 0 | 0 | 2 |
| Oligoastrocytoma | 1 | 0 | 0 | 1 |
| Glioblastoma multiforme | 2 | 0 | 0 | 2 |
| Medulloblastoma | 0 | 2* | 1 | 3 |
| Ependymoma | 0 | 1 | 1* | 2 |
| Hemangioblastoma | 0 | 2 | 0 | 2 |
| Esthesioneuroblastoma | 1 | 0 | 0 | 1 |
| Meningioma | 2 | 0 | 0 | 2 |
| Craniopharyngioma | 0 | 1 | 1 | 2 |
| Ganglioglioma | 1 | 0 | 0 | 1 |
| Duodenum | | | | |
| Adenocarcinoma | 4 | 0 | 0 | 4 |
| Salivary gland | | | | |
| Basal cell adenoma | 1 | 0 | 0 | 1 |
| Myoepithelial tumor | 1 | 0 | 0 | 1 |
| Pleomorphic adenoma | 6 | 0 | 0 | 6 |
| Oncocytoma | | | | |
| Warthin's tumor | 2 | 0 | 0 | 2 |
| Adenoid cystic carcinoma | 3 | 0 | 0 | 3 |
| Basal cell adenocarcinoma | 1 | 0 | 0 | 1 |
| Low grade polymorphous | 1 | 0 | 0 | 1 |
| adenocarcinoma | | | | |
| Mucoepidermoid carcinoma | 2 | 0 | 0 | 2 |
| Thymus | | | | |
| Thymoma | 2 | 0 | 2 | 4 |
| Lung | | | | |
| Adenocarcinoma | 7 | 1* | 0 | 8 |
| Squamous cell carcinoma | 3 | 2* | 0 | 5 |
| Small cell carcinoma | 3 | 1 | 0 | 4 |
| Large cell carcinoma | 3 | 0 | 0 | 3 |
| Neuroendocrine carcinoma | 1 | 0 | 0 | 1 |
| Low grade mucoepidermoid carcinoma | 1 | 0 | 0 | 1 |
| Adenoid cystic carcinoma | 1 | 0 | 0 | 1 |
| Carcinoid tumor | 1 | 0 | 0 | 1 |
| Mesothelioma | 2 | 3 | 0 | 5 |
| Thyroid gland | | | | |
| Papillary carcinoma | 3 | 0 | 0 | 3 |
| Follicular carcinoma | 0 | 0 | 1 | 1 |
| Medullary carcinoma | 2 | 0 | 1 | 3 |
| Follicular adenoma | 2 | 0 | 0 | 2 |
| Lymph node | | | | |
| Follicular lymphoma | 3 | 0 | 0 | 3 |
| Diffuse large B-cell lymphoma | 3 | 0 | 0 | 3 |
| Chronic lymphocytic lymphoma/ | 1 | 0 | 0 | 1 |
| leukemia | | | | |
| Plasmacytoma | 0 | 0 | 1 | 1 |
| T-cell lymphoma, NOS | 0 | 1 | 0 | 1 |

TABLE 5
Soft Tissue Sarcoma

| | Negative | Weak | Strong | N |
|---|---|---|---|---|
| Leiomyosarcoma | 33 | 67 | 47 | 147 |
| Gastrointestinal stromal tumor | 170 | 136 | 108 | 414 |
| Desmoid-type fibromatosis | 34 | 43 | 13 | 90 |

TABLE 6
Normal Tissue

| | Negative | Weak | Strong | N |
|---|---|---|---|---|
| Lung | 7 | 1 | 0 | 8 |
| Liver | 5 | 0 | 0 | 5 |
| Heart | 2 | 0 | 0 | 2 |
| Kidney | 5 | 1 | 0 | 6 |
| Colon | 6 | 0 | 0 | 6 |
| Prostate | 1 | 4 | 2 | 7 |
| Testis (normal) | 2 | 3 | 5 | 10 |
| Testis (atrophic) | 7 | 0 | 0 | 7 |
| Thyroid | 4 | 0 | 0 | 4 |
| Adrenal | 4 | 2 | 2 | 8 |
| Parathyroid | 1 | 0 | 0 | 1 |
| Parotid | 10 | 0 | 0 | 10 |
| Submandibular | 2 | 0 | 0 | 2 |
| Sublingual | 1 | 0 | 0 | 1 |
| Muscle (skeletal) | 7 | 0 | 0 | 7 |
| Artery | 2 | 0 | 0 | 2 |
| Nerve | 1 | 0 | 0 | 1 |
| Pancreas | 3 | 0 | 0 | 3 |
| Ovary | 4 | 0 | 0 | 4 |
| Stomach | 4 | 1 | 0 | 5 |
| Small bowel | 9 | 0 | 0 | 9 |
| Spleen | 0 | 4 | 0 | 4 |
| Esophagus | 3 | 0 | 0 | 3 |
| Fallopian tube | 2 | 0 | 0 | 2 |
| Endometrium | 0 | 0 | 2 | 2 |
| Myometrium | 3 | 0 | 0 | 3 |
| Lymph node | 13 | 0 | 0 | 13 |
| Bladder | 1 | 0 | 0 | 1 |

The normal tissue show that ROR2 is extremely low in most normal tissues, i.e., it's very cancer-specific and anti-ROR2 Tx is likely to have few off-target effects, as the protein is not normally expressed in the organs that were examined.

TABLE 7
Endometrial Tumors

| | Negative | Moderate/Strong | N |
|---|---|---|---|
| Endometroid | 135 | 40 | 175 |
| Serous | 8 | 3 | 11 |
| Endocervical | 33 | 2 | 35 |

TABLE 8
TSC Tumors

| | Negative | Moderate/Strong | N |
|---|---|---|---|
| Angiomyolipoma | 4 | 16 | 20 |
| Lymphangioleiomyomatosis | 1 | 11 | 12 |
| PEComa | 16 | 9 | 25 |

TSC tumors arise in patients with the tuberous sclerosis complex genetic defect. Specifically, these patients get tumors called angiomyolipoma (AML), lymphangioleiomyomatosis (LAM), and perivascular epithelioid cell tumor (PEComa).

### Example 4

### An Anti-ROR2 Monoclonal Antibody Decreases Tumor Growth in a Xenograft Model of Leiomyosarcoma

ROR2-positive LMS05 cells were transduced in vitro with a lentivirus designed to express GFP and luciferase, enabling the use of bioluminescent imaging to monitor tumor engraftment in vivo. 75,000 LMS05 cells were the injected subcutaneously on the backs of 8-week-old NOD/SCID/interleukin (IL)-2Rγ^{null} (NSG) immunodeficient mice. To confirm LMS05 tumor engraftment one week post-injection, bioluminescent activity was visualized in vivo after D-luciferin injection (Biosynth) on an IVIS Spectrum (Caliper Life Sciences) instrument and quantified using Image 4.0 software, as described previously (Willingham et al. (2012) Proc Natl Acad. Sci. USA 109:6662-6667). Total flux (photons/second) values were obtained from each mouse, and mice were matched and randomized into two groups of six animals according to baseline tumor luminescence.

The mice were subsequently treated three times per week via intraperitoneal injection with 200 μg of anti-ROR2 mAb (R&D Systems Human ROR2 MAb, Clone 231509, Mouse IgG2A) or PBS control. After six weeks of treatment, the mice were euthanized and the xenotransplanted tumors were resected and weighed. We found that treatment with anti-ROR2 mAb resulted in a 40% decrease in tumor mass (FIG. 10, Student's t-test, p=0.06), thereby demonstrating that a monoclonal antibody targeting ROR2 can reduce growth of ROR2-positive tumors in vivo and highlighting the use of an ROR2 mAb in cancer therapy.

### Example 5

### An Anti-ROR2 Monoclonal Antibody Decreases ROR2 Signaling

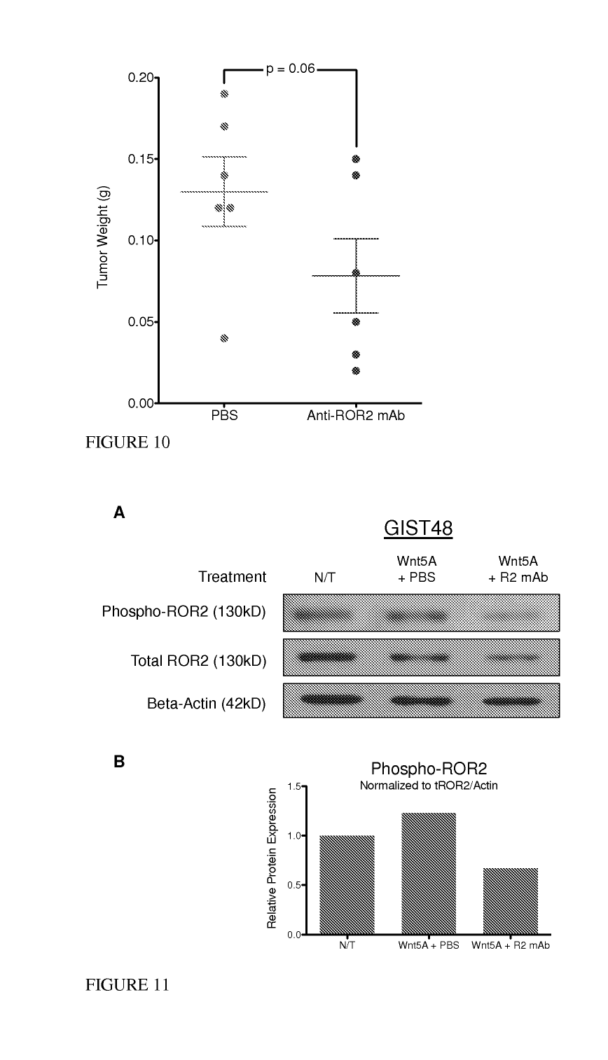

ROR2-positive GIST48 cells were plated in growth medium containing 10% FBS (Invitrogen), allowed to adhere overnight, and then serum starved for 16 h. The cells were then treated with PBS (Invitrogen) or with 400 ng/mL of recombinant human Wnt5A (R&D Systems, Minneapolis, Minn.) in the presence or absence of 1 μg/mL of anti-ROR2 mAb (R&D Systems Human ROR2 MAb, Clone 231509, Mouse IgG2A). Protein lysates were prepared as described previously (Edris et al. (2012) J Pathol 227:223-33) and ROR2 signalling was determined using a phospho-specific ROR2 polyclonal antibody (R&D Systems) and subsequently normalized to total ROR2 and β-Actin levels, as described previously (Edris et al., supra). We found that treatment with ROR2-ligand Wnt5A resulted in an increase in endogenous ROR2 receptor activation as measured by ROR2 phosphorylation, and that this activation was markedly diminished in the presence of the anti-ROR2 mAb, as determined by Western blot and quantified by densitometry using the ImageJ software (FIG. 11A-11B). These results demonstrate that an anti-ROR2 mAb can decrease ROR2-specific activation and downstream signalling, thereby highlighting the therapeutic potential of an ROR2 mAb in blocking the signaling of ROR2-positive cancer cells.

Other embodiments of the invention will be apparent to those skilled in the art from a consideration of the specification or practice of the invention disclosed herein. It is intended that the specification and Examples be considered as exemplary only, with the true scope of the invention being indicated by the following claims.

What is claimed is:

1. A method of inhibiting growth of a cancer cell, the method comprising:
contacting a ROR2 cancer cell with an effective dose of a ROR2 antibody for a period of time sufficient to inhibit growth of the cancer cell.

2. The method of claim 1, wherein the cancer cell is a sarcoma.

3. The method of claim 2, wherein the sarcoma is GIST or leiomyosarcoma.

4. The method of claim 1, wherein the cancer is a carcinoma.

5. The method of claim 4, wherein the carcinoma is a breast carcinoma.

6. The method of claim 1, wherein the cancer cell is in vivo.

7. The method of claim 1, wherein the antibody inhibits growth of the cancer cell by inhibiting activity of ROR2.

8. The method of claim 1, wherein the antibody inhibits growth of the cancer cell by apoptosis, ADCC or CDC.

9. The method of claim 1, wherein the antibody inhibits growth of the cancer cell by delivering a cytotoxic agent.

10. A method of classifying a cancer, comprising the steps of:
providing a suspected GIST or leiomyosarcoma tumor sample;
detecting expression or activity of a ROR2 polypeptide or a gene encoding a ROR2 polypeptide in the sample; and
providing a classification of the tumor based on the results of the detecting step, wherein increased expression of ROR2 relative to a control cell is indicative of a poor prognosis.

11. The method of claim 10 further comprising a step of detecting expression or activity of a gene encoding a KIT or PDGFRA polypeptide in the sample, wherein the classifying step is based on the results of both detecting steps.

12. The method of claim 10 further comprising steps of detecting expression or activity of a gene encoding a KIT polypeptide in the sample; and detecting expression or activity of a gene encoding a PDGFRA polypeptide in the sample, wherein the classifying step is based on the results of all three detecting steps.

13. The method of claims 10-12, wherein the tumor sample is isolated from a subject having a tumor, the method further comprising a step of providing diagnostic, prognostic, or predictive information about the subject based on the results of the classifying step.

14. The method of claims 10-13, wherein the tumor sample is isolated from a subject having a tumor, the method further comprising a step of stratifying the subject for a clinical trial based on the results of the classifying step.

15. The method of claims 10-14, wherein the tumor sample is isolated from a subject having a tumor, the method further comprising a step of selecting a treatment based on the results of the classifying step.

16. The method of claims 10-15, wherein the step of detecting comprises detecting a ROR2 polypeptide or fragment thereof.

17. The method of claim 16, wherein the polypeptide or fragment is detected by performing immunohistochemical analysis on the sample using an antibody that specifically binds to the polypeptide.

18. The method of claims 10-15, wherein the step of detecting comprises detecting a nucleotide molecule encoding a ROR2 polypeptide.

19. The method of claim 18, wherein the nucleotide molecule is detected by in situ hybridization.

20. The method of claims 10-15, wherein the tumor sample is selected from the group consisting of a blood sample, a urine sample, a serum sample, an ascites sample, a saliva sample, a cell, and a portion of tumor tissue.

21. The method of claim 20, wherein the tumor sample is a portion of tumor tissue.

22. A kit for use in classifying GIST or leiomyosarcoma, the kit comprising one or more antibodies for a ROR2 polypeptide; and instructions for use of the kit.

23. The kit of claim 22 further comprising a control slide comprising tumor samples for testing reagents in the kit.

24. The kit of claim 23 further comprising one or more antibodies for a KIT polypeptide and/or a PDGFRA polypeptide, wherein the control slide comprises gastrointestinal stromal tumor samples.


SEQUENCE LISTING (6 sequences)

SEQ ID NO: 1 - DNA, 19 bp, Homo sapiens
ggcagaacccatcctcgtg

SEQ ID NO: 2 - DNA, 19 bp, Homo sapiens
cgactgcgaatccaggacc

SEQ ID NO: 3 - DNA, 21 bp, Homo sapiens
acacctctttccaaacaggcc

SEQ ID NO: 4 - DNA, 21 bp, Homo sapiens
ggattgttaaactcaactctc

SEQ ID NO: 5 - DNA, 19 bp, Homo sapiens
gcacccagcacaatgaaga

SEQ ID NO: 6 - DNA, 20 bp, Homo sapiens
cgatccacacggagtacttg